(19) United States
(12) Patent Application Publication
(10) Pub. No.: US 2009/0075923 A1
(43) Pub. Date: Mar. 19, 2009

(54) Methods of treatment of renal disease

(76) Inventors: Orna Mor, Kiryat Ono (IL); Elena Feinstein, Rehovot (IL)

Correspondence Address: COOPER & DUNHAM, LLP, 30 Rockefeller Plaza, 20th Floor, NEW YORK, NY, 10112, US

(21) Appl. No.: 11/827,198

(22) Filed: Jul. 10, 2007

Related U.S. Application Data
(63) Continuation of application No. 10/365,135, filed on Feb. 12, 2003, now abandoned.
(60) Provisional application No. 60/356,374, filed on Feb. 12, 2002.

(51) Int. Cl. A61K 31/7105; A61P 13/12

(52) U.S. Cl. 514/44

(57) ABSTRACT

The invention is directed to a process of identifying a compound capable of inhibiting the activity of a human Ax1 receptor that comprises contacting the Ax1 receptor or cells expressing the Ax1 receptor with the compound; measuring the Ax1 receptor activity in the presence of the compound; and comparing the activity measured to that measured in the absence of the compound under controlled conditions, wherein a decrease identifies the compound as being capable of inhibiting the activity. Therapeutic and diagnostic applications are also described.

This application claims the benefit of U.S. Provisional Application No. 60/356,374, filed Feb. 12, 2002.

### FIELD OF THE INVENTION

The present invention relates to the identification and isolation of polynucleotide sequences, the expression of which is changed in various renal pathologies, and use of these isolated polynucleotides as probes for diagnosis, for screening of treatment modalities and as target for inactivation in fibrosis in general, and for kidney fibrosis and glomerulosclerosis, hallmarks of diabetic nephropathy, in particular.

### BACKGROUND OF THE INVENTION

Accumulation of extracellular matrix and proliferation of fibroblasts are major hallmarks of fibrosis. Due to secretion of cytokines and growth factors, especially transforming growth factor beta (TGF-β), phenotypic change in fibroblast cells leads to increased deposition of extracellular matrix proteins. Repeated insults trigger up-regulation of tissue inhibitors of matrix metalloproteinases, favoring accumulation of extracellular matrix (Br J Surg 2001 11:1429-1441). Fibrosis is known to occur in many tissues (e.g., kidney, liver, lung, heart) in which injury or other specific stimulus causes acute inflammation at early stages, followed by scar formation and usually culminating in end-stage disease.

Cytokines are critical to a myriad of fundamental homeostatic and pathophysiological processes such as fever, wound healing, inflammation, tissue repair and fibrosis. They play important roles in regulating cell function such as proliferation, migration, and matrix synthesis. It is the balance or net effect of the complex interplay among these mediators and their downstream target proteins that appears to play a major role in regulating the initiation, progression and resolution of wounds and tissue fibrosis.

### Diabetic Nephropathy

Diabetic nephropathy (hallmarks of which are glomerulosclerosis and renal fibrosis) is the single most prevalent cause of end-stage renal disease in the modern world, and diabetic patients constitute the largest population on dialysis. Such therapy is costly and far from optimal. Transplantation offers better outcome but suffers from a severe shortage of donors. More targeted therapies against diabetic nephropathy (as well as against other types of kidney pathologies) are not developed, since molecular mechanisms underlying these pathologies are largely unknown. Identification of a target essential functional gene that is modulated in the disease and affects the severity of the outcome of diabetes nephropathy has a diagnostic as well as therapeutic value.

It is known that many pathological processes in the kidney eventually culminate in similar or identical morphological changes, namely glomerulosclerosis and fibrosis. This means that different types of insults converge on the same single genetic program resulting in the proliferation of fibroblasts and overproduction by them of various protein components of connective tissue—two hallmarks of fibrosis. In addition, thickening of the basal membrane in the glomeruli accompanies interstitial fibrosis and culminates in glomerulosclerosis. Genes encoding proteins that are involved in kidney fibrosis and glomerulosclerosis may be roughly divided into two groups:
1. genes, the expression of which lead to the triggering of these alterations; these may be specific to different pathological conditions.
2. genes, the expression of which are responsible for the execution of the “fibrotic or sclerotic programs”; these may be common to all renal pathologies leading to fibrosis and glomerulosclerosis.

The identification of genes that belong to the second group should contribute to the understanding of molecular mechanisms that accompany fibroblast and mesangial cell proliferation and hypersecretion, and may constitute genetic targets for drug development aimed at preventing renal failure. Application of such drugs is expected to suppress, retard, prevent, inhibit or attenuate progression of fibrosis and glomerulosclerosis.

It is clear that the best way to assess the development of diabetic nephropathy is to characterize gene expression in established animal models of the disease. Examples of such models include (i) fa/fa rats, animals genetically deficient in leptin receptor that develop insulin resistant diabetes (type II diabetes) with progressive diabetic nephropathy, and (ii) GK rats, which are genetically manipulated, NIDDM phenotype rats. Another animal model in which the kidney fibrosis is evident but without a background of diabetes is unilateral ureteral obstruction (UUO) in which interstitial fibrosis is rapid and occurs within days following the obstruction.

Additional aspects of research may be based on an in vitro model system involving culture of human fibroblasts in vitro under conditions mimicking various parameters of the cell microenvironment existing in the diabetic kidney. These include treatment with high concentrations of glucose (modeling hyperglycemia), low concentrations of glucose, hypoxia (both modeling ischemic conditions that develop in the kidney following fibrosis and glomerulosclerosis) and TGF-β (one of the recognized pathogenic factors in fibrosis). Such a model system may complement the animal models in three important aspects:
1. The system is fibroblast-specific; there is none of the interference often found in complex tissues that contain many cell types.
2. The cells are of human origin (unlike the animal models).
3. The insults are specific and of various concentrations and duration, thus enabling the investigation of both acute and chronic responses.

### The Ax1 Receptor

Ax1 is a member of the receptor tyrosine kinase subfamily. It is an integral plasma membrane protein and has the unique structure of the extracellular region that juxtaposes Immunoglobulin-lambda (IgL) and FNIII domains and an intracellular region which contains an intracellular domain, part of which is the kinase domain. It can bind to the vitamin K-dependent protein Gas6, thereby transducing signals into the cytoplasm. The extracellular domain of Ax1 can be cleaved and a soluble extracellular domain of 65 kDa can be released. Cleavage enhances receptor turnover, and generates a partially activated kinase (O'Bryan J P, Fridell Y W, Koski R, Varnum B, Liu E T. (1995) J Biol Chem. 270(2):551-557). However, the function of the cleaved domain is unknown.

Upon interaction with the Gas6 ligand, Ax1 becomes autophosphorylated, and a cascade of signal transduction events takes place. Known to be involved in this cascade are PI3K, AKT, src, Bad, 14-3-3, PLC, ERK, S6K (mitogen-regulated kinase) and STAT (each of these was studied in different cell lines and/or systems).

Gas6, the ligand of Ax1, has a region rich with γ-carboxyglutamic acid (GLA domain) that allows for Ca⁺⁺-dependent binding to membrane phospholipids. Gas6 is a weak mitogen and has an anti-apoptotic effect in NIH3T3 fibroblasts subjected to stress by TNF-induced cytotoxicity, or growth factor withdrawal. In NIH3T3 the binding of Gas6 to Ax1 results in activation of PI3K, AKT, src and Bad.

In mesangial cells, Gas6 was found to have a mitogenic effect, thus demonstrating a possible function in the progression of glomerulosclerosis. Furthermore, it was recently shown (Yanagita M., Ishimoto Y., Arai H., Nagai K., Ito T., Nakano T., Salant D. J., Fukatsu A., Doi T. and Kita T. (2002) The Journal of Clinical Investigation 110 (2) 239-246), that Gas6 is an autocrine growth factor for mesangial cells, and that the anticoagulant wafarin together with the extracellular domain of Ax1 inhibit mesangial cell proliferation by specific blockade of the Gas6-mediated pathway in a mesangial-proliferative model of glomerulonephritis. Gas6 also promotes the survival of endothelial cells and is up-regulated from 6 h-72 h in the balloon-injured rat carotid artery (a model for arterial injury). Angiotensin II, via its AT1 receptor, was shown to increase Ax1 mRNA and protein receptor in vascular smooth muscle cells (Melaragno M G, Wuthrich D A, Poppa V, Gill D, Lindner V, Berk B C, Corson M A. (1998) Circ Res. 83(7):697-704). The AT1 receptor antagonist losartan blocked the stimulatory effect of angiotensin on Ax1 expression. In the 32D myeloid cell line, expression of Ax1 permits aggregation of cells in response to Gas6 stimulation. This response does not require Ax1 kinase activity; thus, it was suggested that aggregation is mediated by a heterotypic intercellular mechanism whereby cell-bound Gas6 interacts with an Ax1 receptor on an adjacent cell.

Transgenic mice expressing the Ax1 receptor under the GM-CSF promoter exhibit phenotypic characteristics associated with non-insulin-dependent diabetes mellitus (NIDDM), including hyperglycemia and hyperinsulinemia, severe insulin resistance, progressive obesity, hepatic lipidosis, and pancreatic islet dysplasia. These mice were shown to express high levels of TNF-α. Ax1 proteolytic cleavage product (extracellular domain (ECD) of Ax1) created a more severe NIDDM phenotype in transgenic mice (Augustine K A, Rossi R M, Van G, Housman J, Stark K, Danilenko D, Varnum B, Medlock E. (1999) J Cell Physiol. 181(3):433-447).

Ax1 has been shown to be involved in cellular adhesion, cell proliferation and regulation of homeostasis in the immune system (Lu Q and Lemke G (2001) Science 293(5528):306-311). Following Ax1 activation, the following phenomena have been observed: inhibition of apoptosis, increase in “normal” cell (non-transformed) survival of fibroblasts and endothelial cells, migration of Vascular Smooth Muscle Cell (VSMC) (inactivation of the Ax1 kinase blocks migration), enhancement of neointima formation in blood vessel wall (Melaragno M G, Fridell Y W, Berk B C. (1999) Trends Cardiovasc Med. (Review) 9(8):250-253) and involvement in lesion formation and the progression of atherosclerosis. Lack of Gas6 in knock out mice results in reduced nephrotoxicity following acute stimulation suggesting that Ax1, as the major ligand for GAS6 may be involved in this process in normal kidneys. Moreover, the mitogenic effect of GAS6 on mesangial cells may be carried out by signalling through Ax1.

What is Known about the Ax1 Gene:
Synonyms of Ax1: UFO, ARK (in mouse)

### Structural Information Relating to the Human Ax1 Gene and Gene Product:

a. Nucleotide Sequence: 5015 bp variant 1 gi:11863122
4986 bp variant 2 gi:11863124

b. open reading frame: 894 aa (461-3145 bp)—variant 1
885 aa (459-3113 bp)—variant 2

c. Protein sequence: 885 aa mw 140 kDa (human) gi:4502335

### Domains: gi:4502335, Performed by SMART:

a. Extracellular region: 1-33 aa: signal peptide
41-136 and 145-224 aa: (Ig)
225-318 and 334-415 aa: 2 FNIII domains.

b. Transmembrane domain: 441-463 aa

c. Intracellular domain: 527-794 aa of SEQ ID NO:4
The intracellular domain contains a tyrosine kinase domain with motif Lys-Trp-Ile-Ala-Ile-Glu-Ser: SEQ ID NO:6 (present in all Tyro3 family members). Note that the intracellular domain has amino acid sequence SEQ ID NO:5.

Homology has been demonstrated to receptor tyrosine kinases of the Tyro3 family that includes besides Ax1 also Tyro3 (named also Sky or RSE), and MER proteins.

Tissue distribution: The inventors of the present invention, found that in mouse, Ax1 is expressed in distinct structures in a broad range of developing tissues in late embryogenesis and in cells forming organ capsules and connective tissue structures in adults.

Disease relevant patterns: Ax1 is a chronic myelogenous leukemia-associated oncogene and is also associated with colon cancer and melanoma.

Expression pattern during embryonic/fetal development: The Ax1 gene is evolutionarily conserved among vertebrate species, and is expressed during development in the mesenchyme.

The proliferation of mesangial cells seems to be an important pathological event that precedes glomerular sclerosis. Mesangial cells produce extracellular matrix and thus contribute to the fibro-sclerotic changes in the diabetic kidney. Gas6 was found to regulate mesangial cell proliferation through Ax1 in experimental glomerulonephritis. Inhibition of Gas6 interaction with Ax1 reduced proteinuria, mesangial cell proliferation, and restored renal function (Yanagita M et al., (1999) J Am Soc Nephrol 10:2503-2509; Yanagita M et al., (2001) Am J Pathol, 158:1423-1432). The following patent publications also relate to Ax1 or other tyrosine kinase receptors: U.S. Pat. No. 5,468,634; U.S. Pat. No. 6,087,144; U.S. Pat. No. 5,538,861; U.S. Pat. No. 5,968,508; U.S. Pat. No. 6,211,142; U.S. Pat. No. 6,235,769; WO 99/49894; WO 00/76309; WO 01/16181 and WO 01/32926.

Nowhere in the background art is it taught or suggested that modulation of the Ax1 receptor is useful for diagnosis and treatment of renal disease or, more specifically, diabetic nephropathy.

### SUMMARY OF THE INVENTION

The main object of the present invention is the identification and isolation of novel genetic targets that may be used for development of drugs to treat fibrosis, as well as for development of diagnostic and prognostic applications. It is a further object of the present invention to identify and isolate novel genetic targets for development of drugs to treat renal disease, and more specifically to treat diabetic nephropathy, and using of such targets as a tool for diagnostic and prognostic applications. It is yet a further object of the present invention to identify and isolate novel genetic targets for development of drugs to treat the hallmarks of diabetic nephropathy, namely glomerulosclerosis and renal fibrosis.

The present invention provides these novel targets for development of novel therapeutic and diagnostic means via large-scale microarray-based analysis of gene expression in nephropathy and more specifically in diabetic nephropathy and kidney fibrosis models in vivo and in vitro. Preferably, the present invention identifies up- or down-regulator (responder) genes for gene therapy, diagnostics and therapeutics that have direct causal relationships between a fibrotic nephropathological disease and its related pathologies. More preferably, the present invention identifies the Ax1 gene as an up-regulator gene in the above-mentioned models.

The present invention further provides a process referred to herein as a screening assay for identifying modulators, i.e., candidate or compounds or agents including but not limited to neutralizing antibodies, peptides, peptido-mimetics, small molecules and other drugs, which bind to Ax1 or have an inhibitory effect on Ax1 expression or on Ax1 activity.

The compound or agent discovered by the above-mentioned screening assay that will inhibit signaling via the Ax1 receptor may be used in diabetic nephropathy to down-regulate mesangial cell proliferation and to slow the pace of or inhibit glomerulosclerosis or to reduce the proliferation of fibroblasts, to inhibit the accumulation of extracellular matrix and to reduce or limit the formation of fibrotic regions in the kidney. Preferably, the present invention identifies up- or down-regulator (responder) genes for gene therapy, diagnostics and therapeutics that have direct causal relationships between a disease and its related pathologies. More preferably, the present invention identifies the Ax1 gene for the above-mentioned uses.

### BRIEF DESCRIPTION OF THE FIGURES

FIG. 1. This Figure demonstrates the endogenous Ax1 expression in response to TGF-β, in a variety of cell lines (Western blot analysis). Lane 1: NRK 49F cells; Lane2: NRK 49F cells with TGF-β 5 ng/ml, 24 hr; Lane 3: Rat1 cells; Lane4: Rat1 cells+TGF-β 5 ng/ml, 24 hr; Lane 5: WI38 cells; Lane 6: HeLa cells; Lane 7: 293 cells.

FIG. 2. This Figure demonstrates that Ax1 protein is up-regulated in rat1 cells in response to TGF-β. Total cell lysates from Rat1 cells exposed to TGF-β stimulation (5 ng/ml for 15 minutes, 30 minutes, 60 minutes and 2 hours) were run on gel and probed with anti Ax1 C20 Ab (Western blot analysis). Samples in the first and last lane are from cells without TGF-β stimulation.

FIG. 3. This Figure demonstrates that TGF-β-dependent induction of Ax1 is accompanied by increase in phosphorylated-Ax1 levels. Rat1 cells exposed to TGF-β stimulation (5 ng/ml for 15 minutes, 30 minutes, 60 minutes and 2 hours) were used for immunoprecipoitation. Ax1 was immunoprecipited with anti Ax1 M-20. Anti phosphotyrosine antibodies were used to monitor ax1 phosphorylation state following TGF-β treatment (5 ng/ml for 15 minutes-2 hr). Samples in the first and last lane are from cells without TGF-β stimulation.

FIG. 4. This Figure demonstrates up regulation of the Ax1 polypeptide following UUO in Rat. Expression of Ax1 protein was monitored in normal SD rat kidneys following UUO model. Lane 1: Ax1 expression in contralateral (cl) non-treated kidneys; Lane 2: Ax1 expression in obstructed kidneys (UUO, for 25 days).

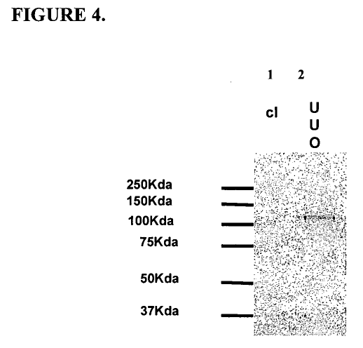

### DETAILED DESCRIPTION OF THE INVENTION

According to the present invention, purified, isolated and cloned nucleic acid sequences, specifically the nucleic acid sequence that encodes the Ax1 receptor, associated with nephropathy and more specifically with diabetic nephropathy and with fibrotic and glomerulosclerotic kidneys and having sequences as specified herein or having complementary or allelic sequence variations thereto, are disclosed. Furthermore, a purified, isolated and cloned nucleic acid sequence associated with nephropathy and having a sequence which encodes SEQ ID NO: 2 and 4 herein is also disclosed. The database provides two transcript variants:

transcript variant 1: NM_{—}021913 GI:11863122

transcript variant 2: NM_{—}001699 GI:11863124

As used herein, the term “Ax1 gene” is defined as any homolog of the Ax1 gene having preferably 90% homology, more preferably 95% homology, and even more preferably 98% homology to the amino acid encoding region of SEQ ID NO:1 and NO:3 or nucleic acid sequences which bind to the Ax1 gene under conditions of highly stringent hybridization, which are well-known in the art (for example Ausubel et al., Current Protocols in Molecular Biology, John Wiley and Sons, Baltimore, Md. (1988), updated in 1995 and 1998). Note that 18 nucleotides upstream of the ATG in both SEQ ID NO: 1 and NO:3 are not in the amino acid encoding region, and many nucleotides downstream of the stop signal are also not in the amino acid encoding region.

As used herein, the term “Ax1” or “Ax1 polypeptide” or “Ax1 receptor” is defined as any homolog of the Ax1 polypeptide having preferably 90% homology, more preferably 95% homology, and even more preferably 98% homology to SEQ ID NO:2, to SEQ ID NO:4, or to SEQ ID NO:5 as either full-length or a fragments or a domain thereof, as a mutant or the polypeptide encoded by a spliced variant nucleic acid sequence, as a chimera with other polypeptides, provided that any of the above has the same or substantially the same biological function as the Ax1 receptor. Ax1 polypeptide, or an Ax1 polypeptide homolog, may be present in different forms, including but not limited to soluble protein, membrane-bound (either in purified membrane preparations or on a cell surface), bead-bound, or any other form presenting Ax1 protein or fragments and polypeptides derived thereof. The Ax1 polypeptide or Ax1 receptor comprises the intracellular domain represented by SEQ ID NO:5.

Particular fragments of the Ax1 polypeptide include amino acids 1-50, 51-100, 101-150, 151-200, 201-250, 251-300, 301-350, 351-400, 401-450, 451-500, 501-550, 551-600, 601-650, 651-700, 701-750, 751-800 and 801-850, of SEQ ID NOS: 2 and 4, and amino acids 851-894 and 851-885 of SEQ ID NOS: 2 and 4 respectively. Further particular fragments of the Ax1 polypeptide include amino acids 25-74, 75-124, 125-174, 175-224, 225-274, 275-324, 325-374, 375-424, 425-474, 475-524, 525-574, 575-624, 625-674, 675-724, 725-774, 775-824 and 825-874 of SEQ ID NOS: 2 and 4 and amino acids 875-894 and 875-885 of SEQ ID NOS: 2 and 4 respectively.

It is also envisaged by the instant invention that inhibition of any other members of the Tyro3 family, which includes Tyro3, Ax1 and Mer, may have therapeutic results similar to those observed by inhibition of Ax1.

Where the sequences are partial sequences, they may be used as markers/probes for genes that are up-regulated in fibrosis. In general these partial sequences which are designated “Expressed Sequence Tags” (ESTs), are markers for the genes actually expressed in vivo, and are ascertained as described herein in the Examples section. Generally, ESTs comprise DNA sequences corresponding to a portion of nuclear encoded mRNA. The EST has a length that allows for polymerase chain reaction (PCR), and is used as a hybridization probe, with a unique designation for the gene with which it hybridizes (generally under conditions sufficiently stringent to require at least 95% base pairing). For a detailed description and review of ESTs and their functional utility see WO 93/00353 which is incorporated herein in its entirety by reference WO 93/00353 further describes how the EST sequences can be used to identify the transcribed genes.

As used herein, a “target molecule” is a molecule with which Ax1 or an Ax1 gene family member binds or interacts or phosphorylates or activates in nature; for example, a molecule on the surface of a cell that expresses Ax1, a molecule on the surface of a second cell, a molecule associated with the internal surface of a cell membrane or a cytoplasmic molecule. An Ax1 target molecule is mainly a component of a signal transduction pathway that facilitates transduction of an extracellular signal from Ax1 (e.g., a signal generated by the binding of a ligand of Ax1 to the membrane-bound Ax1 molecule) through the cell membrane and into the cell. The target, for example, can be a second intercellular protein that mediates downstream signaling from Ax1.

As used herein, the term “compound” is defined as comprising any small chemical molecule, antibodies, neutralizing antibodies, antisense DNA or RNA molecules, siRNA, proteins, polypeptides and peptides including peptido-mimetics and dominant negatives, and expression vectors.

In one embodiment, the invention provides assays for screening candidates or compounds that bind to, modulate the activity of, or modulate the expression level of Ax1. The compounds of the present invention can be obtained using any of the numerous approaches in combinatorial and non-combinatorial library methods known in the art, including biological libraries (proteins, peptides, etc.), spatially addressable parallel solid phase or solution phase libraries, synthetic library methods, and natural product libraries.

The modulator of Ax1 expression (transcription or translation) or polypeptide activity may be inter alia a small chemical molecule which generally has a molecular weight of less than 2000 daltons, more preferably less than 1000 daltons, even more preferably less than 500 daltons. Other modulators may be antibodies preferably neutralizing antibodies or fragments thereof including single chain antibodies, antisense oligonucleotides, antisense DNA or RNA molecules, proteins, polypeptides and peptides including peptido-mimetics and dominant negatives, and expression vectors. These modulators may act as follows: small molecules may affect expression and/or activity; antibodies-only activity; all kinds of antisense—will effect Ax1 expression; dominant negative and peptidomimetics-only activity; expression vectors may be used inter alia for delivery of antisense or dominant-negative.

Approaches have recently been developed that utilize small molecules, which can bind directly to proteins and can be used to alter protein function (for review see B. R. Stockwell, (2000) Nature Reviews/Genetics, 1, 116-125). As mentioned above, low molecular weight organic compounds can permeate the plasma membrane of target cells relatively easily and, therefore, methods have been developed for their synthesis. These syntheses, in turn, have yielded libraries that contain ligands for many proteins. Recent developments have brought a greatly increased variety of creatively selected, novel, small organic molecules that will function as powerful tools for perturbing biological systems. Such small molecules can be used to activate or inactivate specific members of a protein family.

Examples of methods for the synthesis of molecular libraries can be found in the art, for example, in DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. 5,223,409) plasmids (Cull et al. (1992) Proc. Natl. Acad. Sci. (USA) 89:1865-1869) or on bacteriophage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310).

In accordance with another embodiment of the present invention, an assay is a cell-based assay in which cells of mammalian origin are transfected with a kinase active Ax1 construct. The cells are contacted with a compound; the ability of the compound to inhibit Ax1 activity is determined.

In yet another embodiment, the assay is comprised of incubating cells over-expressing active Ax1 with a second molecule preferably an Ax1 target, to form an assay mixture. This assay mixture is then incubated with a compound identified according to any of the screening processes of the present invention, and the ability of the identified compound to inhibit Ax1 activity towards its target is determined.

Thus in this embodiment the ability of the identified compound to interact with Ax1 is determined by measuring the ability of the identified compound to preferentially bind to Ax1 as compared to the Ax1 target molecule i.e. the second compound (i.e. measurement of competitive binding).

In another embodiment, an assay is a cell-based assay comprising contacting cells expressing an Ax1 receptor or fragment thereof, with a compound and determining the ability of the compound to modulate (i.e., stimulate or inhibit) the activity of Ax1. Determining the ability of the compound to modulate the activity of Ax1 can be accomplished, for example, by determining the enzymatic activity of Ax1. The latter can be accomplished directly by following tyrosine phosphorylation of cellular proteins downstream to Ax1 (or Ax1 target molecules) or by a reporter based assay based on measuring, for example, metabolically labeling Ax1-expressing cells with radioactive (either ³²P or ³³P) phosphate and following the accumulation of radioactivity in phosphotyrosine-specific immunoprecipitates of cells stimulated by the Ax1 target molecule or by using fluorescence polarization for the detection of Ax1 activity.

Alternatively, determining the activity of Ax1 can be accomplished indirectly by detecting induction of a cellular second messenger of Ax1 and/or its downstream effectors (i.e., increases in intracellular free Ca²⁺ ion, diacylglycerol production, IP₃ generation, etc.), detecting catalytic/enzymatic activity of the target using an appropriate endogenous or exogenous substrate, detecting the induction of a reporter gene (comprising an Ax1-responsive regulatory element operatively linked to a nucleic acid encoding a detectable marker, e.g., luciferase), or detecting a cellular response, for example, cell survival, cellular differentiation, or cell proliferation.

In yet another embodiment, an assay is a cell-free assay comprising incubating recombinant Ax1, or fragments thereof, with a compound and determining the ability of the compound to bind to Ax1. Binding of the compound to Ax1 can be determined either directly or indirectly as described above. For example, the assay comprises incubating Ax1 with a known compound that binds Ax1, or an Ax1 target molecule, to form an assay mixture. This assay mixture is further incubated with a compound and the ability of the compound to preferentially bind to Ax1 as compared to the known compound (or target molecule) is measured.

Yet, in another embodiment of the present invention, an assay is a cell-free assay comprising incubating Ax1 with a compound and determining the ability of the compound to modulate (e.g., stimulate or inhibit) the activity of Ax1. Determining the ability of the compound to modulate the activity of Ax1 can be accomplished by following auto phosphorylation of Ax1 or by following tyrosine phosphorylation of Ax1 substrates by, for example, performing in vitro kinase assays using radioactively-labeled (either ³²P or ³³P) ATP and measuring the accumulation of radioactivity in the phosphorylated substrate, or by using fluorescence polarization using, for example, the commercially available Molecular Devices® kit.

The cell-free assays of the present invention are compatible with the use of either a soluble form, a membrane-bound form or an immobilized form of Ax1. In the case of cell-free assays comprising the membrane-bound form of Ax1, it may be desirable to utilize a solubilizing agent such that the membrane-bound form of Ax1 is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton™ X-100, Triton™ X-114, 3-[(3-cholamidopropyl)dimethylamino]-1-propane sulfonate (CHAPS), or 3-[(3-cholamidopropyl)dimethylamino]-2-hydroxy-1-propane sulfonate (CHAPSO).

In some of the embodiments of the above assay processes, it may be desirable to immobilize either Ax1 or its target molecule to facilitate separation of complexed from uncomplexed forms of one or both of the proteins, as well as to accommodate automation of the assay. Binding of a compound to Ax1, or interaction of Ax1 with a target molecule in the presence and/or absence of a compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to bind to a matrix. For example, glutathione-S-transferase/Ax1 fusion proteins or glutathione-S-transferase/target fusion proteins can be adsorbed onto glutathione Sepharose beads or glutathione derivatized microtitre plates, which are then combined with the compound and either the non-adsorbed target protein or Ax1, and the mixture incubated under conditions suitable for complex formation. Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix and the level of Ax1 binding or activity determined using standard techniques.

Other techniques for immobilizing proteins on matrices can also be used in the screening assays of the invention. For example, either Ax1 or its target molecule can be immobilized utilizing conjugation of biotin and streptavidin. Biotinylated Ax1 or target molecules can be prepared from biotin-NHS (N-hydroxysuccinimide) using techniques well known in the art (e.g., biotinylation kit, Pierce Chemical, Rockford, Ill.), and immobilized in the wells of streptavidin-coated 96 well plates (Pierce Chemical, Rockford, Ill.). Alternatively, antibodies reactive with Ax1 or target molecules but which do not interfere with binding of Ax1 to its target molecule can be bound to the wells of the plate, and free target or Ax1 trapped in the wells by antibody conjugation. Methods for detecting such complexes, in addition to those described above for the GST-immobilized complexes, include immunodetection of complexes using antibodies reactive with Ax1 or target molecule, as well as enzyme-linked assays which rely on detecting an enzymatic activity of Ax1 or that associated with Ax1 or its target molecule.

In another embodiment, modulators of Ax1 expression are identified in a process wherein cells are contacted with a compound and the expression of Ax1 mRNA or protein in the cell sample is determined. The level of expression of Ax1 mRNA or protein in the presence of the candidate compound is compared to the level of expression of Ax1 mRNA or protein in the absence of the candidate compound. The candidate compound can then be identified as a modulator of Ax1 expression based on this comparison. For example, when expression of Ax1 mRNA or protein is greater in the presence of the candidate compound than in its absence, the candidate compound is identified as a stimulator of Ax1 mRNA or protein expression. Alternatively, when expression of Ax1 mRNA or protein is lower in the presence of the compound than in its absence, the candidate compound is identified as an inhibitor of Ax1 mRNA or protein expression. The level of Ax1 mRNA or protein expression in the cells can be determined by methods described herein for detecting Ax1 mRNA or protein.

A preferred embodiment of the present invention provides for a process of identifying a compound capable of inhibiting the activity of a human Ax1 receptor that comprises the steps of:
(i) contacting the Ax1 receptor or cells expressing the Ax1 receptor with the compound;
(ii) measuring the Ax1 receptor activity in the presence of the compound; and
(iii) comparing the activity measured in step (ii) to that measured in the absence of the compound under controlled conditions, wherein a decrease identifies the compound as being capable of inhibiting the activity.

In one embodiment of the invention, the activity measured in the above mentioned process is tyrosine phosphorylation of a substrate of the Ax1 receptor or auto phosphorylation of the Ax1 receptor. In another embodiment the cells that are contacted with the compound are mesangial cells and the activity measured is proliferation of said mesangial cells or the cells contacted with the compound are renal fibroblasts and the activity measured is proliferation of said renal fibroblasts. In further embodiment, the cells contacted with the compound are renal fibroblasts and the activity measured is collagen deposition in the extracellular matrix of said renal fibroblasts. In a further embodiment, the cells contacted with the compound are renal tubular cells and the activity measured is proliferation of said renal tubular cells. Yet, in another embodiment, the cells contacted with the compound are renal tubular cells and the activity measured is transdifferentiation to myofibroblasts.

In another embodiment of present invention, the cells in the contacting step (i) of the above mentioned process have previously been transfected by the Ax1 gene, either transiently or stably transfected. Yet, in another embodiment, the controlled conditions in step (iii) comprises measurement upon contacting cells which lack an active Ax1 gene. In a further embodiment, the controlled conditions in step (ii) comprise comparison upon contacting similar cells having the absence of an active Ax1 gene or similar cells having a mutated inactive form of the Ax1 gene.

In another embodiment of the present invention, the Ax1 receptor of the above mentioned processes comprises consecutive amino acids, the sequence of which is set forth either in SEQ ID NO:5, or SEQ ID NO:2 or SEQ ID NO:4. In a further embodiment, the Ax1 receptor comprises a biologically active portion of the intracellular domain.

In another embodiment of the present invention, the compound identified according to any of the processes mentioned in the above, inhibits the activity of the Ax1 receptor at least 2-fold, more preferably 5-fold, even more preferably 100-fold and most preferably 200-fold, more effectively than it inhibits the activity of the tyrosine kinase receptors FGFR1, VER4, KIN24, HGFr, met, EGFR, IGF-Ir, InsR and Abl.

Yet, in a further embodiment of the invention, a compound identified according to the processes of the above can be used in the preparation of a medicament for therapy of nephropathy.

In a further embodiment of the invention, prior to contacting the Ax1 receptor or cells expressing the Ax1 receptor with the compound, the Ax1 receptor is contacted with a second compound known to bind Ax1. In another embodiment, either the Ax1 receptor or the second compound are immobilized.

An embodiment of the present invention provides for a process of identifying a compound capable of decreasing the level of an Ax1 gene expression that comprises the steps of:
(i) contacting cells capable of expressing an Ax1 receptor with the compound;
(ii) measuring the expression level of the Ax1 gene in the presence of the compound; and
(iii) comparing the level measured in step (ii) to that measured in the absence of the compound, under controlled conditions, wherein a decrease identifies the compound as being capable of inhibiting the activity.

In another embodiment, the cells in the contacting step (i) of the above mentioned process have been transfected by the Ax1 gene, either transiently or stably transfected. Yet, in another embodiment, the controlled conditions in step (iii) comprises comparison upon contacting identical cells in the absence of the chemical compound. In a further embodiment, the controlled conditions in step (ii) comprises comparison upon contacting similar cells having the absence of an active Ax1 gene or the similar cells having a mutated inactive form of the Ax1 gene. In another embodiment, prior to step (ii), the cells of step (i) are exposed to at least one insult that is related to nephropathy. The insult may be selected from the group consisting of hyperglycemia, hypoxia, low glucose concentration, and TGF-β. In accordance with the invention, the cells exposed to the compound can be selected from the group consisting of mesangial cells, renal fibroblasts, and renal tubular cells. Yet, in a further embodiment of the invention, a compound identified according to the above mentioned process can be used in the preparation of a medicament for therapy of nephropathy.

It is the subject of the present invention further to provide for a method of diagnosing nephropathy in a subject comprising determining, in a sample from the subject, the level of an Ax1 receptor encoding polynucleotide, wherein a higher level of the polynucleotide compared to the level of the polynucleotide in a subject free of nephropathy is indicative of nephropathy. In one embodiment, the diagnosed nephropathy is diabetic nephropathy or kidney fibrosis, and the sample is taken from kidney tissue.

This application is also directed to a process of identifying a compound capable of inhibiting the activity of a human Ax1 receptor by screening a plurality of compounds that comprises the steps of:
(i) contacting the Ax1 receptor or cells expressing the Ax1 receptor with the plurality of compounds;
(ii) measuring the Ax1 receptor activity in the presence of the plurality of compounds;
(iii) comparing the activity measured in step (ii) to that measured in the absence of the plurality of compounds under controlled conditions, wherein a decrease identifies the plurality of compounds as being capable of inhibiting the activity; and
(iv) separately determining which compound or compounds present in the plurality inhibit the activity of a human Ax1 receptor.

In yet another aspect of the invention, Ax1 protein can be used as “bait protein” in a two-hybrid assay or three-hybrid assay (e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins which bind to or interact with Ax1 (“Ax1-binding proteins”) and modulate Ax1 activity. Such Ax1-binding proteins are also likely to be involved in the propagation of signals by Ax1 as, for example, upstream or downstream elements of the Ax1 signaling pathway.

The two-hybrid system is based on the modular nature of most transcription factors, which consist of separable DNA-binding and activation domains. Briefly, the assay utilizes two different DNA constructs. In the first construct, the gene that codes for Ax1 is fused to a gene encoding the DNA binding domain of a known transcription factor (e.g., GAL-4). In the second construct, a DNA sequence obtained from a library of DNA sequences that encodes an unidentified protein (“prey” or “sample”) is fused to a gene that codes for the activation domain of the known transcription factor. If the “bait” and the “prey” proteins are able to interact in vivo, forming an Ax1-dependent complex, the DNA-binding and activation domains of the transcription factor are brought into close proximity. This proximity allows transcription of a reporter gene (e.g., LacZ) which is linked to a transcriptional regulatory site responsive to the transcription factor. Expression of the reporter gene can be detected and cell colonies containing the functional transcription factor can be isolated and used to obtain the cloned gene that encodes the protein that interacts with Ax1.

This invention further pertains to novel agents identified by the above-described screening assays and uses thereof for treatments of renal disease and more specifically for the treatment of nephropathy, especially diabetic nephropathy as described herein.

The present invention further provides a process for identifying a compound capable of decreasing the level of Ax1 gene expression useful for therapy of nephropathy. According to that process cells capable of expressing the Ax1 receptor are contacted with a compound, followed by exposing the cells to at least one insult or pathological parameter that is related to nephropathy. Comparison of the level of Ax1 gene expression to that obtained by a control can indicate the inhibitory effect of said compound on the Ax1 activity.

The present invention further provides transgenic animals and cell lines carrying at least one expressible gene, particularly that encoding the Ax1 receptor, identified by the present invention. The present invention further provides knock-out eucaryotic organisms, in which at least one nucleic acid sequence, as identified by the probes of the present invention and prepared as described below, was knocked out.

The present invention provides a process for discovering drugs for use in treating nephropathy in a patient in need of such treatment. These drugs, in therapeutically effective amounts, will be antagonists of at least one protein, particularly the Ax1 receptor, as encoded by the nucleic acid sequences or as presented by the amino acid sequences identified herein or by the probes of the present invention. Although these drugs are preferentially directed to treatment of kidney fibrosis, they may also be useful for the treatment of other fibrotic diseases, such as liver, lung and heart. These drugs may also be used to treat or prevent restenosis, i.e., to prevent or reduce proliferation of smooth muscle cells. These drugs may also be used as anti-angiogenic drugs for the treatment of cancer and other conditions where preventing or reducing proliferation of endothelial cells is desired.

Any of the screening assays according to the present invention can include a step of identifying the compound (as described above) which tests positive in the assay, and can also include the further step of producing as a medicament that which has been so identified. It can also include steps of improving the compound to increase its desired activity before incorporating the improved compound into a medicament. It is considered that medicaments comprising such compounds are part of the present invention.

The present invention further provides for a process of preparing a composition which comprises:
(i) identifying a compound that inhibits activity of a human Ax1 receptor by at least one of the above processes; and
(ii) admixing said compound with a carrier.

In one embodiment of the invention, the carrier of the above mentioned process is a pharmaceutically effective carrier, and the compound admixed with the carrier is present in a pharmaceutically effective amount.

Additionally, the present invention provides a method of regulating fibrosis-associated pathologies in a patient in need of such treatment by administering to a patient a therapeutically effective amount of at least one antisense (AS) oligonucleotide against the nucleic acid sequences or dominant negative peptide directed against the Ax1 sequences or Ax1 proteins.

As used herein, “negative dominant peptide” refers to a partial cDNA sequence that encodes a part of a protein, i.e., a peptide (Herskowitz I. (1987) Nature (Review) 329(6136):219-222). This peptide can have a function different from that of the protein from which it was derived. It can interact with a wild type protein target and inhibit its activity or it can interact with other proteins and inhibit their activity in response to the wild type target protein. Specifically, negative dominant refers to the ability of a peptide to inhibit the activity of a natural protein normally found in the cell in order to modulate the cellular phenotype, i.e., making the cell more resistant or sensitive to killing. For therapeutic intervention either the peptide itself is delivered as the active ingredient of a pharmaceutical composition or the cDNA can be delivered to the cell utilizing the same methods as for AS delivery.

The antagonist/regulating agent/active ingredient is dosed and delivered in a pharmaceutically acceptable carrier as described herein below. As used herein, the term “antagonist or antagonizing” is understood in its broadest sense. Antagonism can include any mechanism or treatment that results in inhibition, inactivation, blocking or reduction in gene activity or gene product. It should be noted that the inhibition of a gene or gene product may provide for an increase in a corresponding function that the gene or gene product was regulating. The antagonizing step can include blocking cellular receptors for the gene products and can include AS treatment as discussed below.

Many reviews have covered the main aspects of AS technology and its enormous therapeutic potential (Anazodo et al. (1995) Gene 166(2):227-232). There are reviews on the chemical (Crooke S T (1995) Hematol Pathol. (Review) 9(2):59-72; Uhlmann et al. (2000) Methods Enzymol. 313:268-284.), cellular (Wagner R W (1994) Nature (Review) 372(6504):333-335), and therapeutic (Hanania et al. (1995) Am J. Med. (Review) 99(5):537-552; Scanlon et al. (1995) FASEB J. (Review) 9(13):1288-1296; Gewirtz A M (1993) Leuk Lymphoma. 1993; 11 Suppl 1:131-137) aspects of this rapidly developing technology. Within a relatively short time, ample information has accumulated about the in vitro use of AS nucleotide sequences in cultured primary cells and cell lines, as well as the in vivo administration of such nucleotide sequences for suppressing specific processes and changing body functions in a transient manner. AS intervention in the expression of specific genes can be achieved by the use of synthetic AS oligonucleotide sequences (for recent reports see Lefebvre-d'Hellencourt et al. (1995) Eur Cytokine Netw. (Review) 6(1):7-19; Agrawal S (1996) Trends Biotechnol. (Review) 14(10):376-387; Lev-Lehman et al. (1997) Blood 89(10):3644-3653. Instead of an AS sequence as discussed herein above, ribozymes may be utilized. This is particularly necessary in cases where AS therapy is limited by stoichiometric considerations (Sarver et al. (1990) Gene Regulation and Aids, pp. 305-325). Ribozymes can then be used that will target the same sequence. Ribozymes are RNA molecules that possess RNA catalytic ability (see Cech T R (1993) Gene (Review) 135(1-2):33-36) and that cleave a specific site in a target RNA molecule.

The ribozyme type utilized in the present invention is selected as is known in the art. Hairpin ribozymes are now in clinical trial and are the preferred type. In general the ribozyme is from 30-100 nucleotides in length.

Modifications or analogs of nucleotides can be introduced to improve the therapeutic properties of the nucleotides. Improved properties include increased nuclease resistance and/or increased ability to permeate cell membranes.

Nuclease resistance, where needed, is provided by any method known in the art that does not interfere with biological activity of the AS oligodeoxy-nucleotides, cDNA and/or ribozymes as needed for the method of use and delivery (Eckstein F (1985) Annu Rev Biochem. (Review) 54:367-402; Spitzer S and Eckstein F (1988) Nucleic Acids Res. 16(24):11691-11704; Woolf et al. (1990) Nucleic Acids Res. 18(7):1763-1769). Modifications that can be made to oligonucleotides in order to enhance nuclease resistance include modifying the phosphorous or oxygen heteroatom in the phosphate backbone. These include preparing methyl phosphonates, phosphorothioates, phosphorodithioates and morpholino oligomers. One embodiment provides for phosphorothioate bonds linking between the four to six 3′-terminus nucleotide bases. Alternatively, phosphorothioate bonds link all the nucleotide bases. Other modifications known in the literature may be used where the biological activity is retained, but the stability to nucleases is substantially increased.

The present invention also includes all analogs of, or modifications to, an polynucleotide or oligonucleotide of the invention that does not substantially affect the function of the polynucleotide or oligonucleotide. The nucleotides can be selected from naturally occurring or synthetically modified bases. Naturally occurring bases include adenine, guanine, cytosine, thymine and uracil. Modified bases of the oligonucleotides include xanthine, hypoxanthine, 2-aminoadenine, 6-methyl-, 2-propyl- and other alkyl-adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In addition, analogs of nucleotides can be prepared wherein the structures of the nucleotides are fundamentally altered and are better suited as therapeutic or experimental reagents. An example of a nucleotide analog is a peptide nucleic acid (PNA) wherein the deoxyribose (or ribose) phosphate backbone in DNA (or RNA) is replaced with a polyamide backbone similar to that found in peptides. PNA analogs have been shown to be resistant to degradation by enzymes and to have extended lives in vivo and in vitro. Further, PNAs have been shown to bind more strongly to a complementary DNA sequence than to a DNA molecule. This observation is attributed to the lack of charge repulsion between the PNA strand and the DNA strand. Other modifications that can be made to oligonucleotides include polymer backbones, cyclic backbones, or acyclic backbones.

The active ingredients of the pharmaceutical composition can include oligonucleotides that are nuclease resistant, needed for the practice of the invention, or a fragment thereof shown to have the same effect targeted against the appropriate sequence(s) and/or ribozymes. Combinations of active ingredients as disclosed in the present invention can be used, including combinations of AS sequences.

The AS oligonucleotides (and/or ribozymes) and cDNA of the present invention can be synthesized by any method known in the art for ribonucleic or deoxyribonucleic nucleotides. For example, an Applied Biosystems 380B DNA synthesizer can be used. When fragments are used, two or more such sequences can be synthesized and linked together for use in the present invention.

The nucleotide sequences of the present invention can be delivered either directly or with viral or non-viral vectors. When delivered directly the sequences are generally rendered nuclease resistant. Alternatively the sequences can be incorporated into expression cassettes or constructs such that the sequence is expressed in the cell as discussed herein below. Generally the construct contains the proper regulatory sequence or promoter to allow the sequence to be expressed in the targeted cell.

The polypeptides of the present invention may be produced recombinantly (see generally Marshak et al., 1996 “Strategies for Protein Purification and Characterization. A laboratory course manual.” Plainview, N.Y.: Cold Spring Harbor Laboratory Press, 1996) and analogs may be produced by post-translational processing. Differences in glycosylation can provide polypeptide analogs.

As used herein, the term “polypeptide” refers to, in addition to a polypeptide, a peptide and a full protein, as well as a fragment or fragments thereof.

As used herein, “functionally relevant” refers to the biological property of the molecule and in this context means an in vivo effector or antigenic function or activity that is directly or indirectly performed by a naturally occurring polypeptide or nucleic acid molecule. Effector functions include but are not limited to receptor binding, any enzymatic activity or enzyme modulatory activity, any carrier binding activity, any hormonal activity, any activity in promoting or inhibiting adhesion of cells to extracellular matrix or cell surface molecules, or any structural role, as well as having the nucleic acid sequence encode functional protein and be expressible. The antigenic functions essentially mean the possession of an epitope or an antigenic site that is capable of cross-reacting with antibodies raised against a naturally occurring protein. Biologically active analogs share an effector function of the native polypeptide that may, but need not, in addition possess an antigenic function.

In diagnosis, the sample is taken from a bodily fluid or from a tissue, preferably kidney tissue; the bodily fluid is selected from the group of fluid consisting of blood, lymph fluid, ascites, serous fluid, pleural effusion, sputum, cerebrospinal fluid, lacrimal fluid, synovial fluid, saliva, stool, sperm and urine, preferably blood or urine. Measurement of level of the Ax1 polypeptide may be determined by a method selected from the group consisting of immunohistochemistry, western blotting, ELISA, antibody microarray hybridization and targeted molecular imaging. Such methods are well-known in the art, for example for immunohistochemistry: M. A. Hayat (2002) Microscopy, Immunohistochemistry and Antigen Retrieval Methods For Light and Electron Microscopy, Kluwer Academic Publishers; Brown C (1998): “Antigen retrieval methods for immunohistochemistry”, Toxicol Pathol; 26(6): 830-1); for western blotting: Laemmeli UK(1970): “Cleavage of structural proteins during the assembly of the head of a bacteriophage T4”, Nature; 227: 680-685; and Egger & Bienz (1994) “Protein (western) blotting”, Mol Biotechnol; 1(3): 289-305); for ELISA: Onorato et al. (1998) “Immunohistochemical and ELISA assays for biomarkers of oxidative stress in aging and disease”, Ann NY Acad Sci 20; 854: 277-90); for antibody microarray hybridization: Huang (2001) “Detection of multiple proteins in an antibody-based protein microarray system, Immunol Methods 1; 255 (1-2): 1-13); and for targeted molecular imaging: Thomas (2001). Targeted Molecular Imaging in Oncology, Kim et al (Eds)., Springer Verlag, inter alia.

Measurement of level of Ax1 polynucleotide may be determined by a method selected from: RT-PCR analysis, in-situ hybridization, polynucleotide microarray and Northern blotting. Such methods are well-known in the art, for example for in-situ hybridization Andreeff & Pinkel (Editors) (1999), “Introduction to Fluorescence In Situ Hybridization: Principles and Clinical Applications”, John Wiley & Sons Inc.; and for Northern blotting Trayhurn (1996) “Northern blotting”, Proc Nutr Soc; 55(1B): 583-9 and Shifman & Stein (1995) “A reliable and sensitive method for non-radioactive Northern blot analysis of nerve growth factor mRNA from brain tissues”, Journal of Neuroscience Methods; 59: 205-208 inter alia.

This application is also directed to a method of diagnosing nephropathy, preferably diabetic nephropathy or kidney fibrosis, in a subject comprising determining in a sample from the subject the level of an Ax1 receptor polypeptide, wherein a higher level of the polypeptide compared to the level in a subject free of nephropathy is indicative of nephropathy. In preferred embodiments the Ax1 receptor comprises consecutive amino acids, the sequence of which is set forth in SEQ ID NO:5, SEQ ID NO:2 or SEQ ID NO:4. The sample is taken from a bodily fluid, preferably blood or urine.

The above discussion provides a factual basis for the use of the sequences of the present invention to identify nephropathy-regulated genes and provide diagnostic probes. The methods employed and the utility of the present invention are demonstrated by the following non-limiting examples.

### Methods

### General Methods in Molecular Biology

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In situ (In cell) PCR in combination with Flow Cytometry can be used for detection of cells containing specific DNA and mRNA sequences (Testoni et al., 1996, Blood 87:3822.)

### General Methods in Immunology

Standard methods in immunology known in the art and not specifically described are generally followed as in Stites et al (eds), Basic and Clinical Immunology (8th Edition), Appleton & Lange, Norwalk, Conn. (1994) and Mishell and Shiigi (eds), Selected Methods in Cellular Immunology, W.H. Freeman and Co., New York (1980).

### Immunoassays

In general ELISAs, where appropriate, are one type of immunoassay employed to assess a specimen. ELISA assays are well known to those skilled in the art. Both polyclonal and monoclonal antibodies can be used in the assays. Where appropriate other immunoassays, such as radioimmunoassays (RIA) can be used as are known to those skilled in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al., Molecular Cloning: A Laboratory Manual, Cold Springs Harbor, N.Y., 1989.

### Antibody Production

The term “antibody”, as herein defined, includes monoclonal antibodies (Mabs), polyclonal antibodies and also antibody fragments, such fragments having antibody functional activity and that can be prepared from antibodies and include Fab, F(ab′)₂, Fv and scFv prepared by methods known to those skilled in the art (Bird et al. (1988) Science 242:423-426). Antibodies may be monoclonal, polyclonal or recombinant.

Conveniently, antibodies may be prepared against the immunogen or portion thereof, for example, a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art, as described generally in Harlow and Lane (1988), Antibodies: A Laboratory Manual, Cold Spring Harbor Laboratory, Cold Spring Harbor, N.Y., and Borrebaeck (1992), Antibody Engineering—A Practical Guide, W.H. Freeman and Co., NY. Antibody fragments may also be prepared from the antibodies and include Fab, F(ab′)₂, and Fv by methods known to those skilled in the art.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific; that is, the sera can be absorbed against related immunogens so that no cross-reactive antibodies remain in the sera, rendering it monospecific.

For producing monoclonal antibodies the technique involves hyperimmunization of an appropriate donor with the immunogen, generally a mouse, and isolation of splenic antibody-producing cells. These cells are fused to an immortal cell, such as a myeloma cell, to provide a fused cell hybrid that is immortal and secretes the required antibody. The cells are then cultured, in bulk, and the monoclonal antibodies harvested from the culture media for use.

For producing recombinant antibody (see generally Huston et al. (1991) “Protein engineering of single-chain Fv analogs and fusion proteins” in Methods in Enzymology (J J Langone, ed., Academic Press, New York, N.Y.) 203:46-88; Johnson and Bird (1991) “Construction of single-chain Fvb derivatives of monoclonal antibodies and their production in Escherichia coli in Methods in Enzymology (J J Langone, ed.; Academic Press, New York, N.Y.) 203:88-99; Mernaugh and Mernaugh (1995) “An overview of phage-displayed recombinant antibodies” in Molecular Methods In Plant Pathology (R P Singh and U S Singh, eds.; CRC Press Inc., Boca Raton, Fla.: 359-365), messenger RNAs from antibody-producing B-lymphocytes of animals, or hybridoma are reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as is well known in the art. (For a general discussion of conjugation of fluorescent or enzymatic moieties see Johnstone & Thorpe (1982.), Immunochemistry in Practice, Blackwell Scientific Publications, Oxford). The binding of antibodies to a solid support substrate is also well known in the art (for a general discussion, see Harlow & Lane (1988) Antibodies: A Laboratory Manual, Cold Spring Harbor Laboratory Publications, New York; and Borrebaeck (1992), Antibody Engineering—A Practical Guide, W.H. Freeman and Co.). The detectable moieties contemplated with the present invention can include, but are not limited to, fluorescent, metallic, enzymatic and radioactive markers such as biotin, gold, ferritin, alkaline phosphatase, β-galactosidase, peroxidase, urease, fluorescein, rhodamine, tritium, ¹⁴C and iodination.

### Recombinant Protein Purification

For standard purification, See Marshak et al. (1996), “Strategies for Protein Purification and Characterization. A laboratory course manual.” CSHL Press. Specific purification protocols used for the production of Ax1 protein are described in the examples part.

### Transgenic and Knockout Methods

The present invention provides for a transgenic gene and a polymorphic gene animal and cellular (cell line) model, as well as for a knockout model. These models are constructed using standard methods known in the art and as set forth in U.S. Pat. Nos. 5,487,992; 5,464,764; 5,387,742; 5,360,735; 5,347,075; 5,298,422; 5,288,846; 5,221,778; 5,175,385; 5,175,384; 5,175,383; 4,736,866; as well as Burke and Olson (1991) “Preparation of Clone Libraries in Yeast Artificial-Chromosome Vectors” in Methods in Enzymology, 194, “Guide to Yeast Genetics and Molecular Biology”, eds. C. Guthrie and G. Fink, Academic Press, Inc., Chap. 17:251-270; Capecchi (1989) “Altering the genome by homologous recombination”, Science, 244:1288-1292; Davies et al. (1992) “Targeted alterations in yeast artificial chromosomes for inter-species gene transfer”, Nucleic Acids Research, 20 (11): 2693-2698; Dickinson et al. (1993) “High frequency gene targeting using insertional vectors”, Human Molecular Genetics, 2(8):1299-1302; Duff and Lincoln (1995) “Insertion of a pathogenic mutation into a yeast artificial chromosome containing the human APP gene and expression in ES cells”, Research Advances in Alzheimer's Disease and Related Disorders Khalid Iqbal (Editor), James A. Mortimer (Editor), Bengt Winblad (Editor), Henry M. Wisniewski (Editor); Huxley et al. (1991) “The human HPRT gene on a yeast artificial chromosome is functional when transferred to mouse cells by cell fusion”, Genomics, 9:742-750; Jakobovits et al. (1993) “Germ-line transmission and expression of a human-derived yeast artificial chromosome”, Nature, 362: 255-261; Lamb et al. (1993) “Introduction and expression of the 400 kilobase precursor amyloid protein gene in transgenic mice”, Nature Genetics, 5:22-29; Pearson and Choi (1993) Expression of the human b-amyloid precursor protein gene from a yeast artificial chromosome in transgenic mice. Proc. Natl. Acad. Sci. (USA), 90:10578-10582; Rothstein, (1991) “Targeting, disruption, replacement, and allele rescue: integrative DNA transformation in yeast” in Methods in Enzymology, 194, “Guide to Yeast Genetics and Molecular Biology”, eds. C. Guthrie and G. Fink, Academic Press, Inc., NY, Chap. 19:281-301; Schedl et al. (1993) “A yeast artificial chromosome covering the tyrosinase gene confers copy number-dependent expression in transgenic mice”, Nature, 362:258-261; Strauss et al. (1993) “Germ line transmission of a yeast artificial chromosome spanning the murine a₁ (I) collagen locus”, Science, 259:1904-1907. Further, PCT patent applications WO 94/23049, WO 93/14200, WO 94/06908, WO 94/28123 also provide information.

Further one parent strain, instead of carrying a direct human transgene, may have the homologous endogenous gene modified by gene targeting such that it approximates the transgene. That is, the endogenous gene has been “humanized” and/or mutated (Reaume et al. (1996) J Biol Chem. 271(38):23380-23388.). It should be noted that if the animal and human sequences are essentially homologous, a “humanized” gene is not required. The transgenic parent can also carry an overexpressed sequence, either the non-mutant or a mutant sequence and humanized or not as required. Herein, the term “transgene” is therefore used to refer to all these possibilities.

Additionally, cells can be isolated from the offspring that carry a transgene from each transgenic parent and that are used to establish primary cell cultures or cell lines as is known in the art.

Where appropriate, a parent strain will be homozygous for the transgene. Additionally, where appropriate, the endogenous non-transgene in the genome that is homologous to the transgene will be non-expressive. Herein, by the term “non-expressive” is meant that the endogenous gene will not be expressed and that this non-expression is heritable in the offspring. For example, the endogenous homologous gene could be “knocked-out” by methods known in the art. Alternatively, the parental strain that receives one of the transgenes could carry a mutation at the endogenous homologous gene rendering it non-expressed.

### Gene Therapy

“Gene therapy” as used herein refers to the transfer of genetic material (e.g., DNA or RNA) of interest into a host to treat or prevent a genetic or acquired disease or condition phenotype. The genetic material of interest encodes a product (e.g., a protein, polypeptide, peptide, functional RNA, AS) the production of which is desired in vivo. In particular, the use of antisense molecules (anti-Ax1 polynucleotide) in gene therapy may be used in accordance with the anti fibrosis aspect of the invention.

Gene therapy of the present invention can be carried out in vivo or ex vivo. Ex vivo gene therapy requires the isolation and purification of patient cells, the introduction of a therapeutic gene and the introduction of the genetically altered cells back into the patient. A replication-deficient virus such as a modified retrovirus can be used to introduce the therapeutic gene into such cells. For example, mouse Moloney leukemia virus (MMLV) is a well-known vector in clinical gene therapy trials. See, e.g., Boris-Lauerie et al., Curr. Opin. Genet. Dev., 3, 102-109 (1993).

In contrast, in vivo gene therapy does not require isolation and purification of a patient's cells. The therapeutic gene is typically “packaged” for administration to a patient such as in liposomes or in a replication-deficient virus such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. In an alternative embodiment, if the host gene is defective, the gene is repaired in situ (Culver (1998) “Site-Directed recombination for repair of mutations in the human ADA gene” (Abstract) Antisense DNA & RNA based therapeutics, Coronado, Calif.). Another approach is administration of “naked DNA” in which the therapeutic gene is directly injected into the bloodstream or muscle tissue, for example wherein the therapeutic gene is introduced into the target tissue by microparticle bombardment using gold particles coated with the DNA. Gene therapy vectors can be delivered to a subject by methods known in the art, for example, intravenous injection, local administration (see U.S. Pat. No. 5,328,470) or by stereotactic injection (see e.g., Chen et al. (1994) PNAS 91:3054-3057), and as generally described in Sambrook et al., Molecular Cloning: A Laboratory Manual, Cold Springs Harbor Laboratory, New York (1989, 1992); in Ausubel et al., Current Protocols in Molecular Biology, John Wiley and Sons, Baltimore, Md. (1989); in Chang et al., Somatic Gene Therapy, CRC Press, Ann Arbor, Mich. (1995); in Vega et al., Gene Targeting, CRC Press, Ann Arbor, Mich. (1995); Vectors: A Survey of Molecular Cloning Vectors and Their Uses, Butterworths, Boston Mass. (1988); and Gilboa, E et al. (1986) Transfer and expression of cloned genes using retroviral vectors. BioTechniques 4(6):504-512; these vectors may include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

Cell types useful for gene therapy of the present invention include lymphocytes, hepatocytes, myoblasts, fibroblasts, and any cell of the eye such as retinal cells, epithelial and endothelial cells. Preferably the cells are T lymphocytes drawn from the patient to be treated, hepatocytes, any cell of the eye or respiratory or pulmonary epithelial cells. Transfection of pulmonary epithelial cells can occur via inhalation of a neubulized preparation of DNA vectors in liposomes, DNA-protein complexes or replication-deficient adenoviruses. See, e.g., U.S. Pat. No. 5,240,846. For a review of the subject of gene therapy, in general, see the text “Gene Therapy”, August et al. Advances in Pharmacology 40, Academic Press, 1997.

### Delivery of Gene Products/Therapeutics (Compound)

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound or as a pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration, as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein, which treatment has a length proportional to the length of the disease process and drug effectiveness. The doses may be single doses or multiple doses over a period of several days, but single doses are preferred.

When administering the compound of the present invention parenterally, it will generally be formulated in a unit dosage injectable form (e.g., solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (e.g., glycerol, propylene glycol, liquid polyethylene glycol), suitable mixtures thereof, and vegetable oils.

Proper fluidity can be maintained, for example, by the use of a coating such as lecithin, by the maintenance of the required particle size in the case of dispersion and by the use of surfactants. Non-aqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, e.g., parabens, chlorobutanol, phenol and sorbic acid. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicles, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include those presented in U.S. Pat. Nos. 5,225,182; 5,169,383; 5,167,616; 4,959,217; 4,925,678; 4,487,603; 4,486,194; 4,447,233; 4,447,224; 4,439,196 and 4,475,196. Other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques that deliver the compound orally or intravenously and retain the biological activity are preferred.

In one embodiment, the compound of the present invention can be administered initially by intravenous injection to bring blood levels to a suitable level. The patient's blood levels are then maintained by an oral dosage form, although other forms of administration, dependent upon the patient's condition and as indicated above, can be used. The quantity to be administered will vary for the patient being treated and will vary from about 100 ng/kg of body weight to 100 mg/kg of body weight per day and preferably will be from 10 μg/kg to 10 mg/kg

Throughout this application, various publications are referenced by author and year and patents, including United States patents, are referenced by number. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be construed in the nature of description rather than of limitation.

Obviously, many modifications and variations of the present invention are possible in light of the above teachings. It is therefore to be understood that within the scope of the described invention, the invention may be practiced otherwise than as specifically described.

### EXAMPLE 1

### Identification of Ax1 Overexpression by Microarray Hybridization Study

In accordance with the present invention, the microarray hybridization approach was utilized in order to discover genes that are differentially regulated in diabetic nephropathy and kidney fibrosis.

Microarray-based analysis of gene expression was based on the analysis of human fibroblasts subject to selected stimuli resulting in changes in extracellular collagen accumulation and proliferation—the hallmarks of fibrosis. According to the present invention, a specific “Fibrosis” DNA chip was first prepared followed by a microarray hybridization experiments with 19 different types of probes. Analysis of the results was carried out by proprietary algorithms, and analysis of the selected set of genes was performed by using bioinformatics and the scientific literature.

### Preparation of Specific “Fibrosis” DNA Chip

A dedicated human “Fibrosis” DNA chip was prepared according to assignee's SDGI method (PCT Application Publication No. WO 01/75180) from growth-arrested human fibroblasts. Growth arrest was imposed by the treatments presented in Table 1 below:

TABLE 1
Biological material for “Fibrosis” chip preparation

| | Treatment |
|---|---|
| 1 | G1 arrested serum-starved l.p. HFs* |
| 2 | l.p. HFs* 36 hr and 48 hr following 8Gy γ-irradiation |
| 3 | l.p. HFs* 5 days after addition of H₂O₂ 200 μM |
| 4 | l.p. HFs* following UV (growth-arresting dose) |
| 5 | l.p. HFs* 48 hr following Bleomycin treatment 50 ng/ml |
| 6 | l.p. HFs* 48 hr following Etoposide treatment 400 ng/ml |
| 7 | l.p. HFs* 48 hr following Adriamycin treatment 50 ng/ml |
| 8 | Senescent HFs from normal individuals |
| 9 | Senescent HFs from individuals with Werner syndrome |
| 10 | Senescent HFs from individuals with Progeria |

l.p. HF*—low passage human fibroblasts

Unless indicated otherwise, all human fibroblasts (HFs) were at passage 15 prior to treatment. RNA from all treated HFs was prepared, pooled and used for library preparation by the proprietary SDGI method of the assignee. This chip also contained human ESTs coding for genes known to play a part in apoptosis, cytotoxicity and replicative cellular senescence.

### Fibroblast Cultivation

Normal human fetal lung fibroblasts (WI-38, Coriell Cell Repositories) were cultured and sub-cultured in DMEM, supplemented with 10% inactivated fetal bovine serum (FBS), 2 mM L-glutamine, 100 U/ml penicillin, 100 μg/ml streptomycin. Fibroblasts were grown to confluence in 25 cm² tissue flasks and sub-cultured after trypsinization (0.5% trypsin-EDTA in Hank's balanced solution without Ca²⁺ and Mg²⁺) at 37° C. in an atmosphere of 5% CO₂. Two ml of trypsin were added to each flask and incubated for 5 min; then cultures were centrifuged (5 min, 1000 rpm) and fresh medium was added to the pellet. Splitting conditions were 1:4-1:6.

Since the hallmarks of fibrotic disease are fibroblast proliferation and/or enhanced synthesis of extracellular matrix components (mainly collagen), different treatment regimes were used and the rates of both proliferation and collagen synthesis by the treated fibroblasts cultured in vitro was examined.

### Fibroblast Proliferation Assay

The proliferation rate of sub-confluent fibroblasts was evaluated by staining with neutral red (BioRad). Fibroblasts were seeded in 96-well plate (6×10³/well) in 200 μl of supplemented DMEM/10% FBS. After overnight culture, wells were washed twice with supplemented DMEM/2% FBS. Then, either TGF-β (2-20 ng/ml) or deferoxamine mesylate (DFO, which leads to conditions of chemical hypoxia) at a concentration of 100 mM was added in 200 μl of supplemented DMEM/2% FBS for either 16 hours, 24 hours, 72 hours, or 5 days.

In the case of glucose treatments, after overnight culture, cell-containing wells were washed twice with supplemented glucose-free DMEM/2% FBS. Working concentrations of glucose (5.5 mM, 15 mM, 27.5 mM, or 55 mM) were prepared by dissolving stock solution (110 mM) in supplemented DMEM without glucose/2% FBS. Prepared solutions of glucose were added to fibroblast cultures for either 24 or 72 hours.

Upon completion of incubation, cells were stained with 100 μl of 1% neutral red for 2 hours. After washing with cold PBS, fibroblast monolayers were fixed with 200 μl of ethanol-Sorenson buffer solution (1:1) for 10 min. Optical density was measured with an automated spectrophotometer (λ=540 nm).

### Collagen Production Assay

Collagen production by confluent fibroblast monolayers was assessed by [³H]-proline incorporation into collagenous proteins. Fibroblasts were seeded in 24-well tissue culture plates (2×10⁴/well) and grown in 1 ml of supplemented DMEM/10% FBS until confluence.

Confluent fibroblast cultures were incubated with prepared solutions for either 24 or 48 hr. Then [³H]-proline (10 μCi/well) was added and cultures were incubated for an additional 24 hr. At the end of the incubation, medium was decanted and incubated with or without collagenase for 18 hr, followed by precipitation with 50% and 10% TCA. The production of collagen was determined as the difference between total [³H]proline-containing proteins in the sample incubated without collagenase and those left after collagenase digestion. To determine the number of cells in each well, fibroblasts were detached by trypsinization on the last day of the experiment, and counted in a hemocytometer.

Probes for microarray hybridization were derived from these treated fibroblasts. In accordance with the present invention, treatments that are relevant for diabetic nephropathy development were used, such as glucose deprivation or hypoxia (modeling ischemic conditions that develop in fibrotic kidney); high glucose (modeling diabetic hyperglycemia) and TGF-β induction (modeling a fibrotic condition that is characterized by growth factor and cytokine imbalance).

More specifically, human fibroblasts were treated as followed:
1. glucose at 4 different concentrations (5.5, 15, 27.5, or 55 mM) for 24 and 72 hr
2. TGF-β at 2-20 ng/ml, for 24 or 72 hr
3. DFO deferoxamine at a concentration of 100 mM, dissolved in 0.5 ml of DMEM, containing 5% FCS, 50 μg/ml β-aminoproprionitrile, and 50 μg/ml ascorbic acid (modified DMEM). For 24, 48 and 72 hours.

The analysis of proliferation rate of these cultured fibroblasts showed that cultivation of fibroblasts for 24 hrs in glucose-free medium and in 55 mM glucose resulted in a decrease of their proliferation rate by 20% and 30%, respectively, compared to control cultures. Addition of glucose at different concentrations (from 5.5 mM to 27.5 mM) practically did not affect fibroblast proliferation compared to the control. A significant decrease in fibroblast proliferation was observed after addition of DFO (from 20% decrease after 16 hr incubation to 80% decrease after 5 days of treatment). TGF-β, added at concentrations of 2 and 20 ng/ml, led to an increase in the fibroblast proliferation rate by ˜60% after 24 hrs treatment.

As for collagen synthesis rate, all treatments (except for 55 mM glucose) led to increased collagen production by fibroblasts. The most significant effect was observed after addition of TGF-β at concentrations of 2-20 ng/ml, providing enhancement in collagen production by 110-180%.

In the next step, the RNA from these treated fibroblasts was extracted and used for preparation of probes for microarray hybridization. The scheme of hybridization is presented below:

TABLE 2
Hybridization scheme

| Probe name | Dye | PROBE 1 | Probe name | Dye | PROBE 2 |
|---|---|---|---|---|---|
| FG1A | Cy3 | Untreated human fibroblasts- | FG1B | Cy5 | l.p. untreated HFs* |
| FG19A | | Common Normalizing Probe | FG19B | | l.p. untreated HFs* |
| FG18A | | | FG18B | | l.p. HFs* w/o glucose 72 hr |
| FG17A | | | FG17B | | l.p. HFs* TGF-β 20 ng/μl 72 h |
| FG16A | | | FG16B | | l.p. HFs* TGF-β 20 ng/μl 24 h |
| FG15A | | | FG15B | | l.p. HFs* w/o glucose 24 h |
| FG14A | | | FG14B | | l.p. HFs* TGF-β 2 ng/μl 72 h |
| FG13A | | | FG13B | | l.p. HFs* TGF-β 2 ng/ml 24 h |
| FG12A | | | FG12B | | l.p. HFs* 5.5 mM glucose 72 h |
| FG11A | | | FG11B | | l.p. HFs* 5.5 mM glucose 24 h |
| FG10A | | | FG10B | | l.p. HFs* Hypoxia 5 days |
| FG9A | | | FG9B | | l.p. HFs* 55 mM glucose 72 h |
| FG8A | | | FG8B | | l.p. HFs* 55 mM Glucose 24 h |
| FG7A | | | FG7B | | l.p. HFs* Hypoxia 3 days |
| FG6A | | | FG6B | | l.p. HFs* 27.5 mM Glucose 72 h |
| FG5A | | | FG5B | | l.p. HFs* 27.5 mM glucose 24 h |
| FG4A | | | FG4B | | l.p. HFs* hypoxia 16 h |
| FG3A | | | FG3B | | l.p. HFs* 15 mM glucose 72 h |
| FG2A | | | FG2B | | l.p. HFs* 15 mM glucose 24 h |

l.p. HFs*—low passage human fibroblasts

Probe 1 was identical in all hybridization experiments, and was produced with RNA extracted from untreated human fibroblasts (passage 15). This probe served both as a biological control and as a common normalizing probe that allowed comparison of results obtained from different hybridization experiments.

In accordance with the present invention, a total of 19 hybridization experiments were performed. In two hybridization experiments (FG1 and FG19), the common normalizing probe (Probe 1 in all hybridization experiments) was hybridized against itself (i.e., Probe 1 was identical to Probe 2). In general, these hybridization experiments were conducted in order to determine labeling quality and to evaluate the ability of the common normalizing probe to detect most of the cDNA clones printed on the chip.

### Bioinformatics Analysis of Gene Expression Results

The proprietary statistical analysis of the assignee of microarray hybridization results is based on the assumption that changes in gene expression correlate with different physiological and pathological conditions and, in many instances, underlie them. Thus, in a given set of experiments, a certain treatment regime/condition is associated with a particular gene expression profile. Furthermore, we assume that some hierarchy exists among the different (patho) physiological conditions/treatments, i.e., some are more similar than others.

The final goal of such an analysis is to elucidate both specific and general mechanisms underlying complex biological phenomena by comparison of gene expression patterns within a large panel of conditions, each representing some of its aspects. More specifically, in the set of hybridization results generated in accordance with the present invention, we anticipated observing groups of genes that their expression was either common or unique to different types of conditions relevant to diabetic nephropathy (hypoxia, high glucose, TGF-β), and wherein the response to the applied treatment was either acute or chronic.

### Results of Hybridization Analysis

In accordance with the present invention, in human fibroblasts differentially treated in vitro, a set of 46 genes was identified, the activity of which was significantly up-regulated by various types of applied treatments.

The identified gene products fell into nine distinct functional groups:
1. Extracellular matrix proteins and receptors to extracellular matrix proteins;
2. Secreted growth factor interacting proteins and potential growth factor receptors;
3. Signal transduction adaptor proteins;
4. Cytoskeletal proteins (mostly related to actin cytoskeleton function);
5. Ca²⁺-binding proteins;
6. ER-resident proteins;
7. Nuclear import mediators;
8. Proteins involved in RNA and protein synthesis and processing;
9. Novel genes;

The 46 up-regulated genes identified were divided as follows:
(a) 11 were known genes with known functions with recognized involvement in fibrosis (collagens type III and I (α1 and α2), fibronectin, decorin, β-ig-h3, integrin, TIMP3, CD44, smooth muscle actin, and Arp2/3 (Arc34);
(b) 28 were known genes with known function but with previously unknown involvement in fibrosis. Ax1, the subject of the present invention, falls into this category;
(c) 2 were genes coding for proteins with unknown function and unknown involvement in fibrosis, and
(d) 5 were novel genes.

Using the microarray hybridization technique it was found that the expression of Ax1 has been induced by TGF-β treatment of human fibroblasts by at least 2-fold.

### EXAMPLE 2

### Validation of Ax1 as a TGF-β Induced Gene (Expression and Phosphorylation Status) by In Vitro Experiments

In order to verify the chip hybridization results, the response of endogenous Ax1 expression to TGF-β stimulation was monitored by Western blot analysis. Total cellular proteins from various cell lines, (of which Rat1 cell line was also stimulated by TGF-β (5 ng/ml for 24 hr) were extracted, and the expression of Ax1 was analyzed by Western blot analysis. Thirty (30) μg of total cellular lysate were run on an 8% SDS gel.

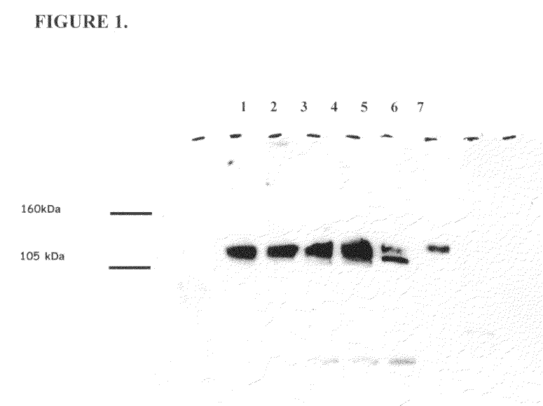

Results showed slight up-regulation following TGF-β stimulation in Rat1 cells (shown in FIG. 1).

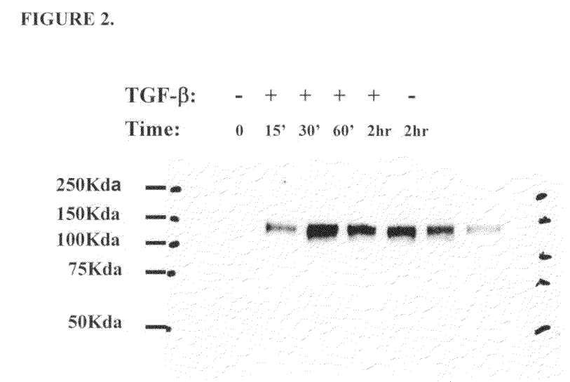

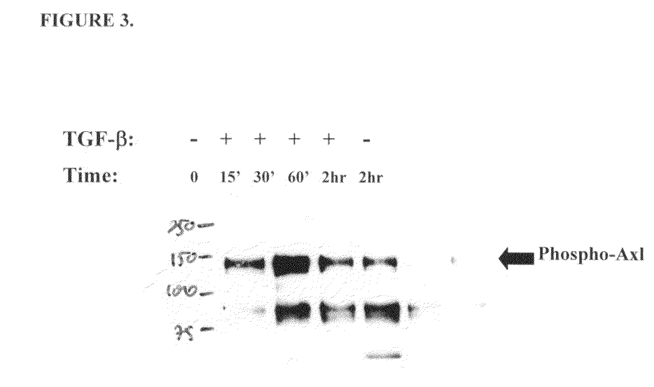

Further experiments were done on Rat1 cells that were serum starved for 24 hr and then stimulated for the indicated time (15 min-2 hr) with 5 ng/ml TGF-β. Results show that indeed TGF-β induces Ax1 protein level (FIG. 2), following 15 min of TGF-β treatment. Increase in its phosphorylation is also observed (FIG. 3) suggesting than in response to TGF-β, Ax1 protein is induced and functionally activated.

### EXAMPLE 3

### Assessment of In Vivo Models for Kidney Fibrosis by Morphology, Immunostaining and In Situ Hybridization

### Morphology

To assess general morphology, paraffin kidney sections were stained by hematoxilin-eosin (HE). The Sirius Red (SR) staining was used to reveal collagen in the sections.

### Immunostaining

Accumulation of interstitial myofibroblasts is regarded as an important initial step in the development of the renal fibrotic process. To reveal myofibroblasts, monoclonal antibody specific to α-smooth muscle actin (clone 1A4) was used for the peroxidase-antiperoxidase (PAP) immunostaining of kidney paraffin sections. The monoclonal antibody PC-10 was used for the immunostaining of proliferating cell nuclear antigen (PCNA). To achieve adequate PCNA immunostaining, de-paraffinized sections were subjected to antigen retrieval procedure before performing PAP staining.

### In Situ Hybridization

³⁵S-labeled riboprobes were synthesized and hybridized to kidney paraffin sections according to standard protocol. After the post-hybridization washing step, sections were air-dried and macro-autoradiography was performed by exposing the slides to X-ray film overnight. For micro-autoradiography, slides were dipped into nuclear track emulsion and stored in darkness at 4° C. Exposed slides were developed after 2-3 weeks and sections were slightly counter-stained with HE and cover-slipped for microscopic examination.

### Probes for In Situ Hybridization

The cDNAs used as the templates for riboprobe synthesis were rat osteopontin cDNA, mouse transforming growth factor β1 cDNA, mouse procollagen α1(I) cDNA and mouse thrombospondin1 cDNA.

### Results:

### ZDF Rats

Samples of 9-month-old ZDF rats (zucker diabetic fatty rats) presented hydronephrotic kidneys with dilated calyces. Microscopically these samples presented a picture of glomerulosclerosis and tubulointerstitial fibrosis. In accordance with these morphological changes, the expression of marker genes as measured by in situ hybridization (osteopontin (OPN), transforming growth factor β1 (TGF-β1) and procollagen α1(I) (Col1)) was significantly changed when compared to normal kidneys. Strong OPN expression was detectable in all tubular structures in both cortex and medulla. The TGF-β1 expression was widespread throughout interstitial cells. Some epithelial cells also showed TGF-β1 expression. Col1 expression was detectable by in situ hybridization in most interstitial cells within the medulla, while cortical expression was “focal”.

Aged fa/fa (Obese Zucker) Rats

Samples of 12-month-old fa/fa rats presented strong glomerulosclerosis and diffuse tubulointerstitial fibrosis throughout the cortex and the medulla. The pattern of marker gene expression corresponded to morphological changes. OPN was expressed by tubular structures in the cortex and the medulla. Multiple interstitial cells expressed TGF-β1. Significantly, multiple foci and single interstitial cells showed strong Col1 expression in both cortex and medulla so that the number of Col1-expressing cells appeared to be higher in fa/fa samples than in ZDF samples.

Interestingly, Col1 expression was not detected in glomeruli of either ZDF or fa/fa rats in spite of the prominent accumulation of collagen, as revealed by Sirius Red staining. This suggested a low steady state level of Col1 mRNA in glomerular cells.

### Aged SD (Normal) Rats

Samples of aged SD rats showed increased accumulation of collagen in glomeruli and interstitial space and increased expression of the marker genes. Significantly, the intensity of fibrotic change varied among samples so that one of four samples studied displayed very few changes compared with young animals; fibrotic change in another sample was confined to “polar” regions, and two samples showed uniform accumulation of collagen and elevated expression of marker genes throughout the sections.

### Goto Kakizaki (GK)/Wistar (Normal) 48-Week-Old Rats

Samples of both GK and Wistar 48-week-old rats showed an accumulation of collagen in glomeruli and interstitial space. This accumulation was more pronounced in the GK samples. Two samples were used for mRNA isolation: C9 and GK9. Both were hybridized to the probe specific for IGFBP4. The in situ hybridization results showed that the GK sample demonstrated elevated expression of this gene.

### Permanent UUO.

A known model for fibrosis was employed—unilateral urether occlusion (UUO). One of the urethers was occluded (see below) and animals were sacrifized 1, 5, 10, 15, 20 and 25 days following occlusion.

Permanent UUO resulted in rapid activation (5 days of UUO) of collagen synthesis by interstitial cells in both medulla and cortex. By 20-25 days of UUO, significant amounts of interstitial collagen were deposited in the interstitial space while glomerular accumulation of collagen was confined to the outer capsule. Thus, permanent UUO samples provided an acute model of tubulointerstitial renal fibrosis without prominent glomerulosclerotic changes.

The above models can be used as model systems for testing the therapeutic efficacy of inhibitors identified via any of the screening systems described

### EXAMPLE 4

### Protocol for Permanent Unilateral Ureteral Obstruction (UUO)

### Test System

Strain: Male Sprague-Dawley rats (9 weeks of age)
Group Size: n=5 for operated rat; n=3 for sham-operated rats
Number of groups: 6 for both sham-operated and operated (i.e., 1 day, 5 days, 10 days, 15 days, 20 days and 25 days post-operation or post-sham operation)

### Procedure

Rats were anaesthetized with Ketamin/Xylazine and the abdominal cavity was opened. After being exposed, the ureter from the right kidney was ligated with a suture over it (UUO). In sham-operated rats, the ureter was exposed but not ligated.

### Study Termination

The study was terminated 24 hr, 5 days, 10 days, 15 days, 20 days and 25 days after the UUO procedure or after the sham operation. At this time point, the rats were sacrificed by exsanguination under CO₂ asphyxiation in order to collect the right kidney. After the capsule was removed the kidney was cut transversely. Half was fixed in 10% buffered formalin and the other half was immediately transferred to an eppendorf tube and frozen in liquid nitrogen for RNA analysis.

### EXAMPLE 5

### Analysis of Expression of the Ax1 Gene in Normal and Fibrotic Human Kidneys

The expression patterns of Ax1 were studied by in situ hybridization using sections from human renal tissue samples. The samples analyzed in this pilot study included:
1. normal human kidney (32 year old female);
2. diabetic human kidney showing signs of glomerulosclerosis and tubulointerstitial fibrosis (62 year old male);
3. renal sclerosis accompanied by vast diffuse fibrosis (56 year old female);
4. rejected kidney transplant showing vascular sclerosis, lymphocyte infiltration, glomerulosclerosis and scarring fibrosis (44 year old female; 2 years after transplantation).

Representative sections of all samples were subjected to trial hybridization to the probe specific to elongation factor 1α mRNA in order to ensure the presence of hybridizable mRNA and to establish the optimal regime of pre-hybridization treatment.

The results show that in normal kidneys the expression of Ax1 is very low. On the other hand in fibrotic kidneys staining indicating higher levels of Ax1 gene in tubular epithelial cells in fibrotic regions within the kidney was observed.

Therefore, these experiments involving in situ hybridization with human fibrotic samples suggested the involvement of the Ax1 gene in the proliferation of tubular epithelial cells in fibrotic regions within the kidney.

### EXAMPLE 6

### Analysis of Expression of the Ax1 Gene in Normal and Fibrotic Rat Kidney Samples

A mouse EST clone (Accession Number: BG293435 gi: 4502194) was used as the template for preparation of a riboprobe complementary to rodent Ax1. The radioactively labeled probe was hybridized to the following sections:
1. Permanent UUO multiblock comprised of control sample fixed 25 days after sham-operation; and samples fixed at 24 hr, 5 d, 10 d and 25 d of UUO (one sample per time point);
2. Rat chronic renal failure sample: kidney of 2 year, 7 month-old rat;
3. ZDF samples: samples of 4.5 (non-fibrotic) and 9 month-old (strongly fibrotic) ZDF kidneys;
4. Fa/fa samples: samples of 3, 6 (non-fibrotic) and 12 month-old (strongly fibrotic) fa/fa kidneys;
5. Rat tissue multiblock.

Analysis of in situ hybridization results demonstrated a low level of Ax1 expression in non-fibrotic samples (sham-operated UUO sample and young ZDF and fa/fa samples). Weak hybridization signal in these samples was localized to glomeruli and single interstitial/perivascular cells. Interestingly, small foci of expression in tubular epithelial cells were observed in young samples of ZDF and fa/fa kidneys. These foci were associated with small accumulations of infiltrating lymphocytes and/or interstitial cells. These latter cell types also showed hybridization signal.

Ureter obstruction resulted in prominent changes in the intensity and pattern of Ax1 hybridization signals so that after 24 hr of UUO, the hybridization signal could be seen above the epithelial lining of thick ascending limbs of Henle's loop and collecting ducts in the outer medulla. The hybridization signal also spread into the cortex where collecting ducts, collecting tubules and distal tubules showed prominent hybridization signal. This pattern of expression suggested rapid activation of Ax1 transcription in the distal part of the nephron in response to obstruction. This pattern of epithelial expression was preserved throughout later time points of UUO. In addition to the epithelial signal, some accumulation of expressing cells could be seen in interstitial cells, beginning at 5 days of UUO. At least some of these interstitial cells could be identified as endothelial.

Samples representing chronic fibrotic models also showed significant changes in the pattern of Ax1 expression. Thus, aged fa/fa samples showed multiple foci of strong Ax1 expression throughout the section. Morphologically, these foci showed prominent signs of tubulointerstitial fibrosis, e.g., accumulation of interstitial cells and proliferation of the tubular epithelium. Both epithelial and interstitial cells displayed hybridization signals. A similar pattern was displayed by the aged ZDF sample. It is noteworthy that multiple foci of tubulointerstitial expression contained tubular profiles with clear signs of atrophy. Atrophic cells showed a hybridization signal for Ax1. The aged ZDF sample was prominent for the presence of areas of inflammatory infiltration. Some of the infiltrating cells showed hybridization signals for Ax1. This feature of Ax1 was observed in 4 out of 7 human fibrotic kidney samples. The Ax1-specific hybridization signal was widespread throughout the section of chronic renal failure sample (2 year, 7 month old rat). As in the rest of the fibrotic samples, expressing structures included atrophic and “proliferating”, interstitial and inflammatory cells.

Thus, results of in situ hybridization studies of the Ax1 gene in rat kidney samples demonstrated a low level of expression in non-fibrotic renal tissue. Pathological samples showed expression of this gene in tubular epithelium and in some interstitial, vascular and inflammatory cells. The pattern of pathological expression was very similar to that found earlier in human fibrotic kidneys. This suggested involvement of the Ax1 gene product in the pathological mechanism common to human and rat renal fibrosis. Significantly, results of the animal study clearly demonstrated that activation of the Ax1 gene followed rapidly after the pro-fibrotic insult (UUO) and persisted at more advanced stages of the process. This suggested that the therapeutic approach aimed at the Ax1 gene product might be applicable at any stage of chronic renal failure. Moreover, rapid activation of Ax1 expression in response to UUO suggested involvement of Ax1 in acute renal failure (this suggestion can be easily tested by in situ hybridization studies of samples obtained from patients with acute renal failure). If so, Ax1-targeted therapy may be beneficial for acute renal failure.

Multiblock analysis shows a rather widespread hybridization signal throughout rat tissues. The hybridization signal is clearly seen in the lamina propria of all compartments of the intestinal tract from esophagus to colon. Morphologically, the positive cells can be identified as fibroblasts and histiocytes/macrophages.

The same two cell types appear to display hybridization signal in connective tissue present in sections of other organs: skin, salivary glands, heart, prostate, portal tracts of liver.

A prominent hybridization signal was observed in the red pulp of spleen. The signal localized mainly to macrophages and to some lymphocytes. A similar pattern of expression (lymphocytes and macrophages) was also found in sinuses within the hillary region of the large lymph node. Another element of the lymphatic system, the thymus, also contains positive lymphocytes. Most of the positive lymphocytes concentrated in the medulla while the cortex scattered contained single positive cells. Scattered cells showing strong hybridization signal could be found in lung sections. Morphology of positive cells suggested that lung expression of the Ax1 gene is confined to the subset of macrophages and lung epithelial (type I) cells. Expressing cells of both types can be found in the alveolar wall and within the alveolar and bronchial lumena. This pattern of lung cell expression suggested that activation of Ax1 expression preceded the “shedding” of these cell types.

In addition to the aforementioned portal tract cells, subsets of liver sinusoidal cells (endothelial, stellate and Kuppfer cells) also showed Ax1 expression. A weak hybridization signal was found in testis. This signal localized to some Sertoli cells and some germ cells within the basal layer of spermatogenic epithelium.

The Ax1-specific probe hybridized also to the sagittal section of the normal rat brain. Results of this hybridization suggested a rather low level of expression in the rat central nervous system. The only prominent site of “concentrated” expression was found in the cerebellum. A weak hybridization signal here localized to the “layer” of cells located at the border between molecular and granular layers. Comparisons with parallel sections stained with anti-MAP2 (neuronal marker) and anti-GFAP (astroglial marker) suggested glial specificity of Ax1 expression in this area. A weak hybridization signal was detected in single endothelial and probably glial cells scattered throughout other areas in the brain tissue.

Thus, in situ hybridization studies suggested rather widespread expression of Ax1 in rat tissues. The sites of expression were the interstitial and connective tissues present in many organs. The level of constitutive expression per cell appeared to be lower than that found in tubulointerstitial components of the fibrotic renal tissue after UUO or in chronic models.

### EXAMPLE 7

### Validation of Ax1 Activity and Relevance to Fibrosis in Cells

To examine the function of Ax1 in vitro several approaches are used:
1. Overexpression of EGFR-Ax1 chimera in cells that are deficient in EGFR (NIH3T3-clone 2.2). Overexpressors were stimulated with EGF. Cellular response relevant for fibrosis is checked (e.g., collagen synthesis, fibroncetin expression).
2. An expression vector harboring the Ax1 full open reading frame (Pires-Ax1) was used to obtain overexpressors of Ax1 in NIH3T3 cells. These cells are further used to analyze the effect of over-expression of Ax1 on cellular fibrosis response. Cellular response is checked (e.g., collagen synthesis, fibronectin expression).
3. Ax1 is also transfected to NRK-49F and NRK-52E cells. The over-expressing cells obtained are used for the collagen assay and integrin expression is measured by FACS. These assays are performed following either TGF-β or GAS-6 stimulation (in NRK-F and NRKE, respectively).

### EXAMPLE 8

### In Vivo Models for “Proof of Concept”

To establish the in vivo functional role of Ax1 in kidney fibrosis and glomeruloscierosis, mice in which the Ax1 gene was disrupted are used. These mice were generated by Profesor Goff in Columbia University and are obtained for the functional validation of Ax1. These mice are being used in order to evaluate kidney function following different models of kidney fibrosis and glomerulosclerosis (e.g., UUO), as compared to their normal counterparts exposed to the same treatment. Subsequently, kidney morphology, smooth muscle actin expression and collagen expression are being evaluated as measures of kidney function.

### EXAMPLE 9

### Immunostaining of Rat Kidney Samples with Anti-AXL Antibodies

Sections of UUO multiblock (including sham operated control, 24 hr, 5 d 10 d, 20 d and 25 d of UUO) and chronic renal failure (2 years 7 months old rat) were immunostained with anti AXL antibodies according to our established protocol (see methods section).

No immunostaining was observed in control (sham operated) sample. UUO samples demonstrated positive immunostaining at 24 hr-25 d. Most prominent staining was observed in apical part of tubular epithelial cells. Starting from 5 d of UUO immunostaining was observed also in interstitial cells. Sample from chronic renal failure kidney also demonstrated prominent tubulointerstitial immunostaining.

### EXAMPLE 10

### Screening Assays

### A. Primary Cell Free In Vitro Assay

Cell Free Assay Based on the Kinase Domain (hCytoAx1) of Ax1 Protein

A fluorescence polarization (FP)-based assay was developed for HTS of chemical libraries to identify small molecule inhibitors of Ax1 tyrosine kinase activity. The assay is based on detecting changes in fluorescence polarization (FP) that occur as a result of substrate tyrosine phosphorylation. In this assay, the substrate phosphorylated phosphorylated by Ax1 (competitor) competes for the binding of a fluorescein-labeled phosphopeptide (tracer) to a phosphotyrosine-specific antibody (pY-Ab). The unbound tracer displays low polarization, while its complex with the phosphotyrosine-specific antibody displays high polarization values due to restricted fluorophore rotation. Addition of a competitor to the tracer-Ab complex therefore results in fluorescence polarization decrease, which can be detected. Among the advantages of the fluorescence polarization technique for HTS are the relative insensitivity to changes in fluorescence intensity due to auto-fluorescence of chemical library components or their quenching effects. Additionally, FP is a homogenous technique that requires no separation of assay components prior to measurement.

Recombinant hCytoAx1 (cytoplasmic domain of the receptor tyrosine kinase, aa 495-894) was produced in insect cells (SF9 cells). For purification NiNTA matrix was used. 3 different substrates were tested as potential Ax1 substrates in the assay:

Peptide 1—Biot-PDEILYVNMDE (major Ax1 autophosphorylation site)
Peptide 2—Biot-LSKKIYNGDYYR (Ax1 activation loop peptide)
PGT—Poly(Glu:Tyr) (4:1)—a universal, commonly-used tyrosine kinase substrate

hCytoAx1 from insect cells was immobilized on beads. The beads bound protein was subjected to an in vitro fluorescence polarization-based tyrosine kinase assay using poly(Glu:Tyr) as substrate and measurement of fluorescence polarization was performed. Activity of the immobilized protein was determined. Use of peptide 1 and peptide 2 is under investigation. Activity of a soluble purified protein is also being examined.

As an alternative to production of Ax1 protein from insect cells, Ax1 is also produced from bacteria expressing the protein. The recombinant hCyto Ax1 (cytoplasmic domain of the receptor tyrosine kinase, aa 495-894) was cloned.

To this end, 3 constructs were made:

GST—cytoAx1
GST—cyto Ax1-His
GST cyto Ax1 K567R (“kinase dead”)—as control.

All constructs showed high expression in bacteria. Glutathione affinity resin, or Ni NTA affinity resin followed by Glutathione affinity resin are used for purification to ensure specificity of hCytoAx1 preparation (devoid of other kinases). The purified protein from bacteria is used for the in vitro assay utilizing the same substrates and protocol described above for the insect cells derived Ax1 protein.

Cell Free Assay Based Full Length hAx1 Protein

The DELFIA® method (Wallac™/PerkinElmer®) based on dissociation of enhanced time-resolved fluorometric assay and enabling high sensitivity with wide dynamic range is employed for screening of hAx1 inhibitors in cell free assay. It is based on the tyrosine phosphorylation of substrate peptide by hAx1.

The method was established. The peptide used was—biotin-KKIYNGDYYRQGR (derived from Ax1 activation loop). hAx1-c-Myc protein (full length hAx1 with c-term myc tag) was expressed in 293 cells. Following cell lysis hAx1-c-Myc was immunoprecipitated with the 9B11 (anti-c-Myc tag antibody) and protein G-Sepharose. Immunocomplexs were used for in vitro kinase reaction in kinase assay buffer, 200 uM ATP and with 0.5 uM biotinylated peptide. Kinase reaction (1 hr) was stopped by the addition of EDTA and DELFIA® assay was performed. Our results demonstrate high activity of immobilized hAx1-c-Myc towards its substrate in this assay. Activity of soluble protein from 293 cells is under analysis

### B. Secondary Cell Based Assay

To evaluate the activity of Ax1 in the presense of inhibitors in a cell system, several approaches were taken.

The first was based on EGFR-hAx1 chimera (extracellular domain of Ax1 replaced by EGFR extracellular domain), using transient approach and STAT 3 for the reporter assay (STAT3 is a downstream target of Ax1). The readout of the assay was luminescence (Stop & Glo®/dual Luciferase® assay-Promega).

Cell lines used were NIH/3T3 (2.2) (devoid of endogenous EGFR expression) and 293T.

These were co-transfected with EGFR-hAx1 chimera, STAT 3—Firefly Luciferase reporter (pSTAT 3-TA luc-Stratagene®) as reporter of induction of Ax1 activity (TA-Luc vector served as control) and Renilla Luciferase (pRL-TK—Promega) to ensure specificity of signal generated by STAT 3. Cells transfected with EGFR-hAx1 kinase-dead (KD) mutant chimera, pSTAT 3-TA luc-Stratagene® and pRL-TK—Promega served as specificity control.

24 h post transfection the medium was replaced with “starvation” medium (DMEM with 0.5% BSA) for additional 24 hrs. Serum starvation protocol was employed in order to minimize the possibility that presence of EGF in the serum may cause EGFR-hAx1 chimera chimera aggregation, leading to its activation. Cells were activated with EGF (10 ng/ml) for 3 hrs (in serum-free medium) and then lysed. A sample from the cell lysates incubated with Firefly luciferase substrate followed by Renilla luciferase substrate (Stop & Glo®/dual Luciferase® assay-Promega).

The results showed that transiently transfected EGFR-hAx1 chimera displayed autophosphorylation while the EGFR-hAx1 chimera kinase-dead (KD) mutant chimera transfected cells did not suggesting that Ax1 is active in the context of EGFR-hAx1 chimera. Ax1 inducible activation by EGF is being optimized by using different serum starvation protocols and optimization of transfection parameters. Following optimization the transient transfection protocol of EGFR-hAx1 chimera with the STAT3-luciferase reproter system is used for cell based assay following stimulation of Ax1 activity by EGF.

An alternative approach for cell based assay relies also on STAT3 reporter-based assay but unlike the first approach—stably-transfected EGFR-hAx1 chimera cell clones showing autophosphorylation and EGF-inducible response are used. Both 293 and NIH3T3 cells are used to produce stable clones of EGFR-hAx1 chimera and STAT 3—Firefly Luciferase reporter system is used as for the transient approach. 293 and NIH3T3 stably transfected with EGFR-hAx1 kinase-dead (KD) mutant chimera are used as control for specificity. These are also evaluated for Ax1 activity in STAT3 reporter based assay as described above

A third alternative approach to EGFR-hAx1 chimera based bioassay (using EGF for stimulation), bioassay using the full length hAXL stimulated with GAS6 is evaluated.

NIH/3T3 (2.2) were transfected and stable clones expressing hAXL and hAXL inactive kinase mutant were generated. These showed no constitutive Ax1 tyrosine phosphorylation.

In the assay the stable clone is transiently transfected with the STAT 3—fire fly Luciferase reporter (pSTAT 3-TA luc—Stratagene) and Renilla Luciferase (pRL-TK—Promega). Following transfection, GAS6 is used for stimulation of Ax1 activity which is measured by luminescence (Stop & Glo®/dual Luciferase® assay—Promega).

1-35. (canceled)

36. A method of treatment of renal disease associated with expression of a gene in a patient in need of such treatment which comprises intravenous administration to the patient of a therapeutically effective amount of siRNA which inhibits the gene.

37. The method of claim 36, wherein the renal disease is selected from kidney fibrosis, renal failure and nephropathy.

38. The method of claim 38, wherein the nephropathy is diabetic nephropathy.

39. The method of claim 38, wherein the renal disease is renal failure.


SEQUENCE LISTING (6 sequences)

SEQ ID NO: 1 - DNA, 5015 bp, Homo sapiens
gagtggagttctggaggaatgtttaccagacacagagcccagagggacagcgcccagagcccagatagagagacacggcctcactggctcagcaccagggtccccttccccctcctcagctccctccctggcccctttaagaaagagctgatcctctcctctcttgagttaacccctgattgtccaggtggcccctggctctggcctggtgggcggaggcaaagggggagccaggggcggagaaagggttgcccaagtctgggagtgagggaaggaggcaggggtgctgagaaggcggctgctgggcagagccggtggcaagggcctcccctgccgctgtgccaggcaggcagtgccaaatccggggagcctggagctggggggagggccggggacagcccggcccgctgccccctcccccgctgggagcccagcaacttctgaggaaagtttggcacccatggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcaccccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccaggggaagcacagccagtccaccagctggtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcctgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcgggggccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccagggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctgagacaaccctccacctggtactccctctcaggatccaagctaagcactgccactgggggaaactccaccttcccactttcccaccccacgccttatccccacttgcagccctgtcttcctacctatcccacctccatcccagacaggtccctggccttctctgtgcagtagcatcaccttgaaagcagtagcatcaccatctgtaaaaggaaggggttggattgcaatatctgaagccctcccaggtgttaacattccaagactctagagtccaaggtttaaagagtctagattcaaaggttctaggtttcaaagatgctgtgagtctttggttctaaggacctgaaattccaaagtctctaattctattaaagtgctaaggttctaaggcctacttttttttttttttttttttttttttttttttttgcgatagagtctcactgtgtcacccaggctggagtgcagtggtgcaatctcgcctcactgcaaccttcacctaccgagttcaagtgattttcctgccttggcctcccaagtagctgggattacaggtgtgtgccaccacacccggctaatttttatatttttagtagagacagggtttcaccatgttggccaggctggtctaaaactcctgacctcaagtgatctgcccacctcagcctcccaaagtgctgagattacaggcatgagccactgcactcaaccttaagacctactgttctaaagctctgacattatgtggttttagattttctggttctaacatttttgataaagcctcaaggttttaggttctaaagttctaagattctgattttaggagctaaggctctatgagtctagatgtttattcttctagagttcagagtccttaaaatgtaagattatagattctaaagattctatagttctagacatggaggttctaaggcctaggattctaaaatgtgatgttctaaggctctgagagtctagattctctggctgtaaggctctagatcataaggcttcaaaatgttatcttctcaagttctaagattctaatgatgatcaattatagtttctgaggctttatgataatagattctcttgtataagatcctagatcctaagggtcgaaagctctagaatctgcaattcaaaagttccaagagtctaaagatggagtttctaaggtccggtgttctaagatgtgatattctaagacttactctaagatcttagattctctgtgtctaagattctagatcagatgctccaagattctagatgattaaataagattctaacggtctgttctgtttcaaggcactctagattccattggtccaagattccggatcctaagcatctaagttataagactctcacactcagttgtgactaactagacaccaaagttctaataatttctaatgttggacacctttaggttctttgctssattctgcctctctaggaccatggttaagagtccaagaatccacatttctaaaatcttatagttctaggcactgtagttctaagactcaaatgttctaagtttctaagattctaaaggtccacaggtctagactattaggtgcaatttcaaggttctaaccctatactgtagtattctttggggtgcccctctccttcttagctatcattgcttcctcctccccaactgtgggggtgtgcccccttcaagcctgtgcaatgcattagggatgcctcctttccgcaggggatggacgatctcccacctttcgggccatgttgcccccgtgagccaatccctcaccttctgagtacagagtgtggactctggtgcctccagaggggctcaggtcacataaaactttgtatatcaacgaaaaaaa

SEQ ID NO: 2 - protein, 894 aa, Homo sapiens [sequence omitted]

SEQ ID NO: 3 - DNA, 4986 bp, Homo sapiens
gagtggagttctggaggaatgtttaccagacacagagcccagagggacagcgcccagagcccagatagagagacacggcctcactggctcagcaccagggtccccttccccctcctcagctccctccctggcccctttaagaaagagctgatcctctcctctcttgagttaacccctgattgtccaggtggcccctggctctggcctggtgggcggaggcaaagggggagccaggggcggagaaagggttgcccaagtctgggagtgagggaaggaggcaggggtgctgagaaggcggctgctgggcaaagccggtggcaagggcctcccctgccgctgtgccaggcaggcagtgccaaatccggggagcctggagctggggggagggccggggacagcccggccctgccccctcccccgctgggagcccagcaacttctgaggaaagtttggcacccatggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctaagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcctgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccagggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctgagacaaccctccacctggtactccctctcaggatccaagctaagcactgccactggggaaaactccaccttcccacttttccaccccacgccttatccccacttgcagccctgtcttcctacctatcccacctccatcccagacaggtccctccccttctctgtgcagtagcatcaccttgaaagcagtagcatcaccatctgtaaaaggaaggggttggattgcaatatctgaagccctcccaggtgttaacattccaagactctagagtccaaggtttaaagagtctagattcaaaggttctaggtttcaaagatgctgtgagtctttggttctaaggacctgaaattccaaagtctctaattctattaaagtgctaaggttctaaggcctacttttttttttttttttttttttttttttttttttgcgatagagtctcactgtgtcacccaggctggagtgcagtggtgcaatctcgcctcactgcaaccttcacctaccgagttcaagtgattttcctgccttggcctcccaagtagctgggattacaggtgtgtgccaccacacccggctaatttttatatttttagtagagacagggtttcaccatgttggccaggctggtctaaaactcctgacctcaagtgatctgcccacctcagcctcccaaagtgctgagattacaggcatgagccactgcactcaaccttaagacctactgttctaaagctctgacattatgtggttttagattttctggttctaacatttttgataaagcctcaaggttttaggttctaaagttctaagattctgattttaggagctaaggctctatgagtctagatgtttattcttctagagttcagagtccttaaaatgtaagattatagattctaaagattctatagttctagacatggaggttctaaggcctaggattctaaaatgtgatgttctaaggctctgagagtctagattctctggctgtaaggctctagatcataaggcttcaaaatgttatcttctcaagttctaagattctaatgatgatcaattatagtttctgaggctttatgataatagattctcttgtataagatcctagatcctaagggtcgaaagctctagaatctgcaattcaaaagttccaagagtctaaagatggagtttctaaggtccggtgttctaagatgtgatattctaagacttactctaagatcttagattctctgtgtctaagattctagatcagatgctccaagattctagatgattaaataagattctaacggtctgttctgtttcaaggcactctagattccattggtccaagattccggatcctaagcatctaagttataagactctcacactcagttgtgactaactagacaccaaagttctaataatttctaatgttggacacctttaggttctttgctssattctgcctctctaggaccatggttaagagtccaagaatccacatttctaaaatcttatagttctaggcactgtagttctaagactcaaatgttctaagtttctaagattctaaaggtccacaggtctagactattaggtgcaatttcaaggttctaaccctatactgtagtattctttggggtgcccctctccttcttagctatcattgcttcctcctccccaactgtgggggtgtgcccccttcaagcctgtgcaatgcattagggatgcctcctttccgcaggggatggacgatctcccacctttcgggccatgttgcccccgtgagccaatccctcaccttctgagtacagagtgtggactctggtgcctccagaggggctcaggtcacataaaactttgtatatcaacgaaaaaaa

SEQ ID NO: 4 - protein, 885 aa, Homo sapiens [sequence omitted]

SEQ ID NO: 5 - protein, 268 aa, Homo sapiens
  PEPTIDE (1)..(268)
VALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDL

SEQ ID NO: 6 - protein, 7 aa, Homo sapiens
  PEPTIDE (1)..(7)
KWIAIES